(19) United States
(12) Patent Application Publication
(10) Pub. No.: US 2025/0252865 A1
(43) Pub. Date: Aug. 7, 2025

(54) SYSTEMS AND METHODS FOR USING ARTIFICIAL INTELLIGENCE AND MACHINE LEARNING TO GENERATE A VIRTUAL COACH

(71) Applicant: BlueForge Alliance, Bryan, TX (US)

(72) Inventor: Arnold Kravitz, St. Petersburg, FL (US)

(21) Appl. No.: 19/187,286

(22) Filed: Apr. 23, 2025

Related U.S. Application Data
(63) Continuation-in-part of application No. 18/394,298, filed on Dec. 22, 2023.
(60) Provisional application No. 63/607,354, filed on Dec. 7, 2023.

(51) Int. Cl. G09B 5/06; A61F 9/06; G09G 3/00

(52) U.S. Cl. CPC G09B 5/065; A61F 9/06; G09G 3/002

(57) ABSTRACT

A system includes a vocational mask configured to be worn by a user, the vocational mask including a user interface configured to present, to the user, one or more graphical elements associated with performing a work task, and a computing device communicatively coupled to the user interface. The computing device is configured to cause the user interface to display the one or more graphical elements that include a virtual coach, receive performance data indicative of performance, by the user, of the work task while wearing the vocational mask, and control at least one display characteristic of the virtual coach based on the performance data.

### CROSS-REFERENCES TO RELATED APPLICATIONS

This application is a continuation-in-part of and claims the benefit of and priority U.S. patent application Ser. No. 18/394,298, filed Dec. 22, 2023, titled “SYSTEMS AND METHODS FOR USING A VOCATIONAL MASK WITH A HYPER-ENABLED WORKER,” which claims the benefit of U.S. Patent Application Ser. No. 63/607,354, filed Dec. 7, 2023, titled “SYSTEMS AND METHODS FOR USING A VOCATIONAL MASK WITH A HYPER-ENABLED WORKER,” the entire disclosures of which are hereby incorporated by reference for all purposes.

### TECHNICAL FIELD

This disclosure relates to enabling workers to perform vocations. More specifically, this disclosure relates to systems and methods for using a vocational mask with a hyper-enabled worker.

### BACKGROUND

People use various tools and/or equipment to perform various vocations. For example, a welder may use a welding mask and/or a welding gun to weld an object. The welder may participate in training courses prior to welding the object. A master welder may lead the training courses to train the welder how to properly weld. In some instances, the master welder may be located at a physical location that is remote from where a student welder is physically located.

### SUMMARY

A system includes a vocational mask configured to be worn by a user, the vocational mask including a user interface configured to present, to the user, one or more graphical elements associated with performing a work task, and a computing device communicatively coupled to the user interface. The computing device is configured to cause the user interface to display the one or more graphical elements that include a virtual coach, receive performance data indicative of performance, by the user, of the work task while wearing the vocational mask, and control at least one display characteristic of the virtual coach based on the performance data.

In other features, the user interface is configured to present the one or more graphical elements via a virtual retinal display. The at least one display characteristic includes at least one of a persona, expression, posture, clothing, color, movement, size, and position of the virtual coach. The computing device is configured to obtain a performance score based on the performance data and control the at least one display characteristic based on the performance score. The computing device is configured to obtain a plurality of performance values based on the performance data and calculate the performance score based on the plurality of performance values.

In other features, the computing device is configured to assign respective weights to the plurality of performance values and calculate the performance score as a weighted average of the performance values. The computing device is configured to implement a machine learning model trained to calculate the performance score based on the performance data. The machine learning model is configured to generate a control output to control the virtual coach based on the performance score and improvement data, and the improvement data indicates improvement of performance of the user at the work task.

In other features, the work task is a welding task. The performance data indicates at least one of a straightness of a weld, a thickness of the weld, temperature, travel speed, gas presence, an amount of welding material used, porosity of the weld, and penetration of the weld.

In one embodiment, a method (e.g., a computer-implemented method) includes steps corresponding to functions performed by any system described herein.

In one embodiment, a tangible, non-transitory computer-readable medium stores instructions that, when executed, cause a processing device to perform any operation of any method disclosed herein.

In one embodiment, a system includes a memory device storing instructions and a processing device communicatively coupled to the memory device. The processing device executes the instructions to perform any operation of any method disclosed herein.

In one embodiment, a vocational mask is configured to implement functions of any system or method described herein.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIGS. 1 illustrates a system architecture according to certain embodiments of this disclosure;

FIG. 2 illustrates a component diagram for a vocational mask according to certain embodiments of this disclosure;

FIG. 3 illustrates bidirectional communication between communicatively coupled vocational masks according to certain embodiments of this disclosure;

FIG. 4 illustrates an example of projecting an image onto a user's retina via a virtual retinal display of a vocational mask according to certain embodiments of this disclosure;

FIG. 5 illustrates an example of an image including instructions projected via a virtual retinal display of a vocational mask according to certain embodiments of this disclosure;

FIG. 6 illustrates an example of an image including a warning projected via a virtual retinal display of a vocational mask according to certain embodiments of this disclosure;

FIG. 7 illustrates an example of a method for executing an artificial intelligence agent to determine certain information projected via a vocational mask of a user according to certain embodiments of this disclosure;

FIG. 8 illustrates an example of a method for transmitting instructions for performing a task via bidirectional communication between a vocational mask and a computing device according to certain embodiments of this disclosure;

FIG. 9 illustrates an example of a method for implementing instructions for performing a task using a peripheral haptic device according to certain embodiments of this disclosure;

FIG. 10 illustrates an example computer system according to embodiments of this disclosure;

FIGS. 11A-11C illustrate an example user interface configured to display a virtual avatar or coach according to embodiments of the present disclosure;

FIG. 12 illustrates steps of an example method of controlling the user interface and virtual coach of FIGS. 11A-11C according to embodiments of the present disclosure;

FIG. 13 is a block diagram illustrating one embodiment of a system including a an artificial intelligence agent; and

FIG. 14 is a block diagram of an example artificial intelligence agent.

### NOTATION AND NOMENCLATURE

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer or section from another region, layer or section. Terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), solid state drives (SSDs), flash memory, or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### DETAILED DESCRIPTION

The following discussion is directed to various embodiments of the disclosed subject matter. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

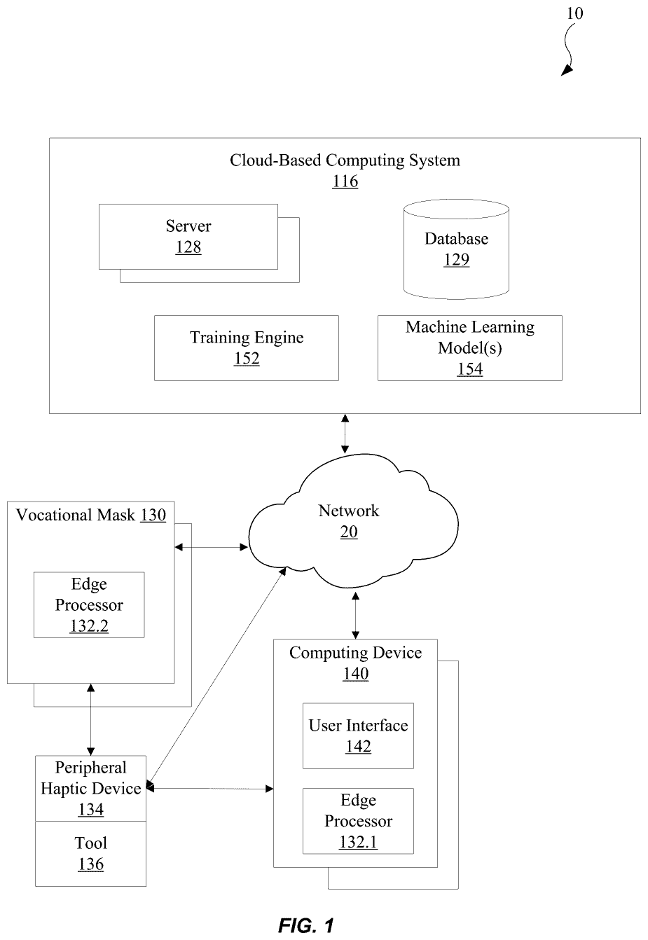

FIGS. 1 through 14 discussed below, and the various embodiments used to describe the principles of this disclosure in this patent document are by way of illustration only and should not be construed in any way to limit the scope of the disclosure.

Some of the disclosed embodiments relate to one or more artificial intelligent enhanced vocational tools for workers to use to perform a job, task, and/or vocation. In some embodiments, the vocational tools may be in the form of a vocational mask that projects work instructions using imagery, animation, video, text, audio, and the like. The vocational tools may be used by workers to enhance the efficiency and proficiency of performing professional and vocational tasks, such as but not limited to supply chain operations, manufacturing and warehousing processes, product inspection, coworker and master-apprentice bidirectional collaboration and communication with or without haptic sensory feedback, other telepresence, and the like.

Some of the disclosed embodiments may be used to collect data, metadata, and multiband video to aid in product acceptance, qualification, and full lifecycle product management. Further, some of the disclosed embodiments may aid a failure reporting, analysis, and corrective action system, a failure mode, effects, and criticality analysis system, other sustainment and support activities and tasks to accommodate worker dislocation and multi-decade lifecycle of some products.

In one embodiment, a vocational mask is disclosed that employs bidirectional communication to include voice and imagery and still and audio video imagery recording with other colleagues over a distance. The vocational mask may provide virtual images of objects to a person wearing the vocational mask via a display (e.g., virtual retinal display). The vocational mask may enable bidirectional communications with collaborators and/or students (e.g., in a master-apprentice relationship). Further, the vocational mask may enable bidirectional audio, visual, and haptic communication to provide a master-apprentice relationship. The vocational mask may include multiple electromagnetic spectrum and acoustic sensors/imagers. The vocational mask may also provide multiband audio and video sensed imagery to a wearer of the vocational mask. A master-apprentice relationship may include virtually any teacher-student relationship or other type of relationship in which a wearer of the vocational mask carries out a task while receiving instructions or directions from another user via another computing device that is operatively coupled (e.g., via a network) to the vocational mask. Examples include, but are not limited to, an apprentice carrying out a task within a skilled trade under instruction of a master, a resident carrying out a medical procedure under instruction of a licensed doctor, and so on.

The vocational mask may be configured to provide display capabilities to project images onto one or more irises of the wearer to display alphanumeric data and graphic/animated work instructions, for example. The vocational mask may also include one or more speakers to emit audio related to work instructions, such as those provided by a master trained user, supervisor, collaborator, teacher, etc.

The vocational mask may include an edge-based processor that executes an artificial intelligence agent. The artificial intelligence agent may be implemented in computer instructions stored on one or more memory devices and executed by one or more processing devices (e.g., edge processor or processors). The artificial intelligence agent may be trained to perform one or more functions, such as but not limited to (i) perception-based object and feature identification, (ii) cognition-based scenery understanding, to identify material and assembly defects versus acceptable features, and (iii) decision making to aid the wearer and to provide relevant advice and instruction in real-time or near real-time to the wearer of the vocational mask. The data that is collected may be used for inspection and future analyses of product quality, product design, and the like. Further, the collected data may be stored for instructional analyses and providing lessons, mentoring, collaboration, and the like.

The vocational mask may include one or more components (e.g., processing device, memory device, display, etc.), interfaces, and/or sensors configured to provide sensing capabilities to understand hand motions and use of a virtual user interface (e.g., keyboards) and other haptic instructions. The vocational mask may include a haptic interface to allow physical bidirectional haptic sensing and stimulation via the bidirectional communications to other users and/or collaborators using a peripheral haptic device (e.g., a welding gun).

In some embodiments, the vocational mask may be in the form of binocular goggles, monocular goggles, finishing process glasses (e.g., grind, chamfer, debur, sand polish, coat, etc.), or the like. The vocational mask may be attached to a welding helmet. The vocational mask may include an optical bench that aligns a virtual retinal display to one or more eyes of a user. Certain types of data (e.g., video and image) may be projected from the virtual retinal display into the retinal of a user. The vocational mask may include a liquid crystal display welding helmet, a welding camera, an augmented reality/virtual reality headset, etc.

The vocational mask may augment projections by providing augmented reality cues and information to assist a worker (e.g., welder) with contextual information, which may include setup, quality control, procedures, training, and the like. Further, the vocational mask may provide a continuum of visibility from visible spectrum (arc off) through high-intensity/ultraviolet (arc on). Further, some embodiments include remote feedback and recording of images and bidirectional communications to a trainer, supervisor, mentor, master user, teacher, collaborator, etc. who can provide visual, auditory, and/or haptic feedback to the wearer of the vocational mask in real-time or near real-time.

In some embodiments, the vocational mask may be integrated with a welding helmet. In some embodiments, the vocational mask may be a set of augmented reality/virtual reality goggles worn under a welding helmet (e.g., with external devices, sensors, cameras, etc. appended for image/data gathering). In some embodiments, the vocational mask may be a set of binocular welding goggles or a monocular welding goggle to be worn under or in lieu of a welding helmet (e.g., with external devices, sensors, cameras, etc. appended to the goggles for image/data gathering). In some embodiments, the vocational mask may include a mid-band or long wave context camera displayed to the user and monitor.

In some embodiments, information may be superpositioned or superimposed onto a display without the user (e.g., worker, student, etc.) wearing a vocational mask. The information may include work instructions in the form of text, images, alphanumeric characters, video, etc. The vocational mask may function across both visible light (arc off) and high intensity ultraviolet light (arc on) conditions. The vocational mask may natively or in conjunction with other personal protective equipment provide protection against welding flash. The vocational mask may enable real-time or near real-time two-way communication with a remote instructor or supervisor. The vocational mask may provide one or more video, audio, and data feeds to a remote instructor or supervisor. The vocational mask and/or other components in a system may enable recording of all data and communications. The system may provide a mechanism for replaying the data and communications, via a media player, for training purposes, quality control purposes, inspection purposes, and the like. The vocational mask and/or other components in a system may provide a mechanism for visual feedback from a remote instructor or supervisor. The vocational mask and/or other components in a system may provide a bidirectional mechanism for haptic feedback from a remote instructor or supervisor.

Further, the system may include an artificial intelligence simulation generator that generates task simulations to be transmitted to and presented via the vocational mask. The simulation of a task may be transmitted as virtual reality data to the vocational mask which includes a virtual reality headset and/or display to playback the virtual reality data. The virtual reality data may be configured based on parameters of a physical space in which the vocational mask is located, based on parameters of an object to be worked on, based on parameters of a tool to be used, and the like.

Turning now to the figures, FIG. 1 depicts a system architecture 10 according to some embodiments. The system architecture 10 may include one or more computing devices 140, one or more vocational masks 130, one or more peripheral haptic devices 134, and/or one or more tools 136 communicatively coupled to a cloud-based computing system 116. Each of the computing devices 140, vocational masks 130, peripheral haptic devices 134, tools 136, and components included in the cloud-based computing system 116 may include one or more processing devices, memory devices, and/or network interface cards. The network interface cards may enable communication via a wireless protocol for transmitting data over short distances, such as Bluetooth, ZigBee, NFC, etc. Additionally, the network interface cards may enable communicating data over long distances, and in one example, the computing devices 140, the vocational masks 130, the peripheral haptic devices 134, the tools 136, and the cloud-based computing system 116 may communicate with a network 20. Network 20 may be a public network (e.g., connected to the Internet via wired (Ethernet) or wireless (WiFi)), a private network (e.g., a local area network (LAN) or wide area network (WAN)), or a combination thereof. Network 20 may also include a node or nodes on the Internet of Things (IoT). The network 20 may be a cellular network.

The computing devices 140 may be any suitable computing device, such as a laptop, tablet, smartphone, smartwatch, ear buds, server, or computer. In some embodiments, the computing device 140 may be a vocational mask. The computing devices 140 may include a display capable of presenting a user interface 142 of an application. In some embodiments, the display may be a laptop display, smartphone display, computer display, tablet display, a virtual retinal display, etc. The application may be implemented in computer instructions stored on the one or more memory devices of the computing devices 140 and executable by the one or more processing devices of the computing device 140. The application may present various screens to a user. For example, the user interface 142 may present a screen that plays video received from the vocational mask 130. The video may present real-time or near real-time footage of what the vocational mask 130 is viewing, and in some instances, that may include a user's hands holding the tool 136 to perform a task (e.g., weld, sand, polish, chamfer, debur, paint, play a video game, etc.). Additional screens may be presented via the user interface 142.

In some embodiments, the application (e.g., website) executes within another application (e.g., web browser). The computing device 140 may also include instructions stored on the one or more memory devices that, when executed by the one or more processing devices of the computing devices 140 perform operations of any of the methods described herein.

In some embodiments, the computing devices 140 may include an edge processor 132.1 that performs one or more operations of any of the methods described herein. The edge processor 132.1 may execute an artificial intelligence agent to perform various operations described herein. The artificial intelligence agent may include one or more machine learning models that are trained via the cloud-based computing system 116 as described herein. The edge processor 132.1 may represent one or more general-purpose processing devices such as a microprocessor, central processing unit, or the like. More particularly, the edge processor 132.1 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The edge processor 132.1 may also be one or more special-purpose processing devices such as an application specific integrated circuit (ASIC), a field programmable gate array (FPGA), a digital signal processor (DSP), network processor, or the like.

In some embodiments, the vocational mask 130 may be attached to or integrated with a welding helmet, binocular goggles, a monocular goggle, glasses, a hat, a helmet, a virtual reality headset, a headset, a facemask, or the like. The vocational mask 130 may include various components as described herein, such as an edge processor 132.2. In some embodiments, the edge processor 132.2 may be located separately from the vocational mask 130 and may be included in another computing device, such as a server, laptop, desktop, tablet, smartphone, etc. The edge processor 132.2 may represent one or more general-purpose processing devices such as a microprocessor, central processing unit, or the like. More particularly, the edge processor 132.2 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The edge processor 132.2 may also be one or more special-purpose processing devices such as an application specific integrated circuit (ASIC), a field programmable gate array (FPGA), a digital signal processor (DSP), network processor, or the like.

The edge processor 132.2 may perform one or more operations of any of the methods described herein. The edge processor 132.2 may execute an artificial intelligence agent to perform various operations described herein. The artificial intelligence agent may include one or more machine learning models that are trained via the cloud-based computing system 116 as described herein. For example, the cloud-based computing system 116 may train one or more machine learning models 154 via a training engine 152, and may transmit the parameters used to train the machine learning model to the edge processor 132.2 such that the edge processor 132.2 can implement the parameters in the machine learning models executing locally on the vocational mask 130 or computing device 140. The machine learning models may be trained to monitor various aspects of a virtual reality session between, e.g., an apprentice and a master, and may further be trained to monitor properties of tasks carried out by the apprentice (e.g., a weld, a solder joint, etc.). Based on this monitoring, an artificial intelligence agent using these machine learning models may adjust instructions/directions provided to the apprentice.

The edge processor 132.2 may include a data concentrator that collects data from multiple vocational masks 130 and transmits the data to the cloud-based computing system 116. The data concentrator may map information to reduce bandwidth transmission costs of transmitting data. In some embodiments, a network connection may not be needed for the edge processor 132.2 to collect data from vocational masks and to perform various functions using the trained machine learning models 154.

The vocational mask 130 may also include a network interface card that enables bidirectional communication with any other computing device 140, such as other vocational masks 130, smartphones, laptops, desktops, servers, wearable devices, tablets, etc. The vocational mask 130 may also be communicatively coupled to the cloud-based computing system 116 and may transmit and receive information and/or data to and from the cloud-based computing system 116. The vocational mask 130 may include various sensors, such as position sensors, acoustic sensors, haptic sensors, microphones, temperature sensors, accelerometers, and the like. The vocational mask 130 may include various cameras configured to capture audio and video. The vocational mask 130 may include a speaker to emit audio. The vocational mask 130 may include a haptic interface configured to transmit and receive haptic data to and from the peripheral haptic device 134. The haptic interface may be communicatively coupled to a processing device (e.g., edge processor 132.2) of the vocational mask 130.

In some embodiments, the peripheral haptic device 134 may be attached to or integrated with the tool 136. In some embodiments, the peripheral haptic device 134 may be separate from the tool 136. The peripheral haptic device 134 may include one or more haptic sensors that provide force, vibration, touch, and/or motion sensations to the user, among other things. The peripheral haptic device 134 may be used to enable a person remote from a user of the peripheral haptic device 134 to provide haptic instructions to perform a task (e.g., weld, shine, polish, paint, control a video game controller, grind, chamfer, debur, etc.). The peripheral haptic device 134 may include one or more processing devices, memory devices, network interface cards, haptic interfaces, etc. In some embodiments, the peripheral haptic device 134 may be communicatively coupled to the vocational mask 130, the computing device 140, and/or the cloud-based computing system 116.

The tool 136 may be any suitable tool, such as a welding gun, a video game controller, a paint brush, a pen, a utensil, a grinder, a sander, a polisher, a gardening tool, a yard tool, a glove, medical tools, a brazing torch, a soldering iron, a mechanical tool such as a wrench or pliers, or the like. The tool 136 may be handheld such that the peripheral haptic device 134 is enabled to provide haptic instructions for performing a task to the user holding the tool 136. In some embodiments, the tool 136 may be wearable by the user. The tool 136 may be used to perform a task. In some embodiments, the tool 136 may be located in a physical proximity to the user in a physical space.

In some embodiments, the cloud-based computing system 116 may include one or more servers 128 that form a distributed computing architecture. The servers 128 may be a rackmount server, a router computer, a personal computer, a portable digital assistant, a mobile phone, a laptop computer, a tablet computer, a camera, a video camera, a netbook, a desktop computer, a media center, any other device capable of functioning as a server, or any combination of the above. Each of the servers 128 may include one or more processing devices, memory devices, data storage, and/or network interface cards. The servers 128 may be in communication with one another via any suitable communication protocol. The servers 128 may execute an artificial intelligence (AI) engine and/or an AI agent that uses one or more machine learning models 154 to perform at least one of the embodiments disclosed herein. The cloud-based computing system 116 may also include a database 129 that stores data, knowledge, and data structures used to perform various embodiments. For example, the database 129 may store multimedia data of users performing tasks using tools, communications between vocational masks 130 and/or computing devices 140, virtual reality simulations, augmented reality information, recommendations, instructions, and the like. The database 129 may also store user profiles including characteristics particular to each user. In some embodiments, the database 129 may be hosted on one or more of the servers 128.

In some embodiments the cloud-based computing system 116 may include a training engine 152 capable of generating the one or more machine learning models 154. The machine learning models 154 may be trained to identify perception-based objects and features using training data that includes labeled inputs of images including certain objects and features mapped to labeled outputs of identities or characterizations of those objects and features. The machine learning models 154 may be trained determine cognition-based scenery to identify one or more material defects, one or more assembly defects, one or more acceptable features, or some combination thereof using training data that includes labeled input of scenery images of objects including material defects, assembly defects, and/or acceptable features mapped to labeled outputs that characterize and/or identify the material defects, assembly defects, and/or acceptable features. The machine learning models 154 may be trained to determine one or more recommendations, instructions, or both using training data including labeled input of images (e.g., objects, products, tools, actions, etc.) and tasks to be performed (e.g., weld, grind, chamfer, debur, sand, polish, coat, etc.) mapped to labeled outputs including recommendations, instructions, or both.

The one or more machine learning models 154 may be generated by the training engine 152 and may be implemented in computer instructions executable by one or more processing devices of the training engine 152 and/or the servers 128. To generate the one or more machine learning models 154, the training engine 152 may train the one or more machine learning models 154. The one or more machine learning models 154 may also be executed by the edge processor 132 (132.1, 132.2). The parameters used to train the one or more machine learning models 154 by the training engine 152 at the cloud-based computing system 116 may be transmitted to the edge processor 132 (132.1, 132.2) to be implemented locally at the vocational mask 130 and/or the computing device 140.

The training engine 152 may be a rackmount server, a router computer, a personal computer, a portable digital assistant, a smartphone, a laptop computer, a tablet computer, a netbook, a desktop computer, an Internet of Things (IoT) device, any other desired computing device, or any combination of the above. The training engine 152 may be cloud-based, be a real-time software platform, include privacy software or protocols, and/or include security software or protocols. To generate the one or more machine learning models 154, the training engine 152 may train the one or more machine learning models 154.

The one or more machine learning models 154 may refer to model artifacts created by the training engine 152 using training data that includes training inputs and corresponding target outputs. The training engine 152 may find patterns in the training data wherein such patterns map the training input to the target output and generate the machine learning models 154 that capture these patterns. Although depicted separately from the server 128, in some embodiments, the training engine 152 may reside on server 128. Further, in some embodiments, the database 129, and/or the training engine 152 may reside on the computing devices 140.

As described in more detail below, the one or more machine learning models 154 may comprise, e.g., a single level of linear or non-linear operations (e.g., a support vector machine [SVM]) or the machine learning models 154 may be a deep network, i.e., a machine learning model comprising multiple levels of non-linear operations. Examples of deep networks are neural networks, including generative adversarial networks, convolutional neural networks, recurrent neural networks with one or more hidden layers, and fully connected neural networks (e.g., each neuron may transmit its output signal to the input of the remaining neurons, as well as to itself). For example, the machine learning model may include numerous layers and/or hidden layers that perform calculations (e.g., dot products) using various neurons.

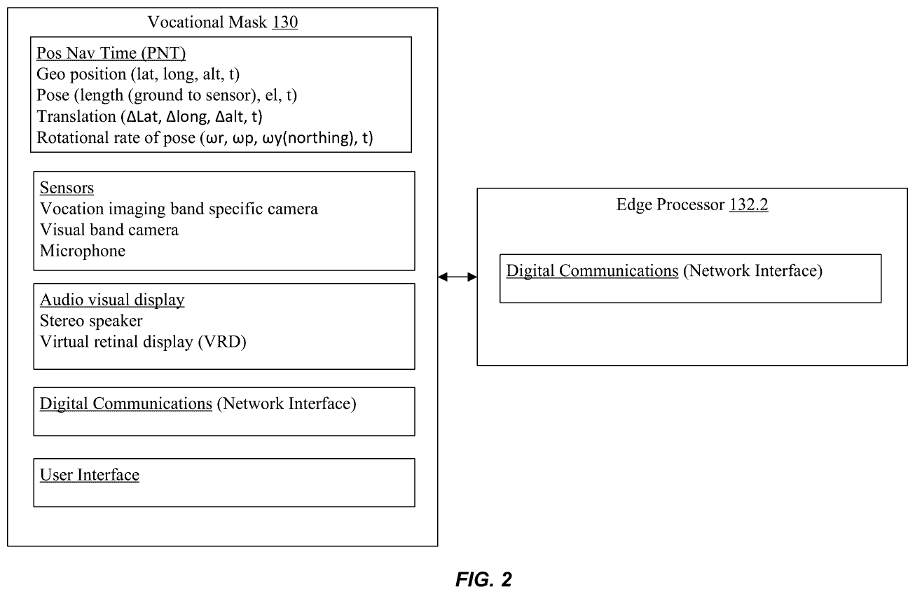

FIG. 2 illustrates a component diagram for a vocational mask 130 according to certain embodiments of this disclosure. The edge processor 132.2 is also depicted. In some embodiments, the edge processor 132.2 may be included in a computing device separate from the vocational mask 130, and in some embodiments, the edge processor 132.2 may be included in the vocational mask 130.

The vocational mask 130 may include various position, navigation, and time (PNT) components, sensors, and/or devices that enable determining the geographical positon (latitude, longitude, altitude, time), pose (length (ground to sensor), elevation, time), translation (delta in latitude, delta in longitude, delta in altitude, time), the rotational rate of pose (ω_r, ω_p, ω_y (northing), t), and the like, where ω_r represents the roll rate, which is the angular velocity about the longitudinal axis of the vocational mask 130, ω_p represents the pitch rate, which is the angular velocity about the lateral axis of the vocational mask 130, ω_y (northing) represents the yaw rate, which is the angular velocity about the vertical axis of the vocational mask 130, referenced with respect to the northing direction, and t represents the time at which these rotational rates are measured.

In some embodiments, the vocational mask 130 may include one or more sensors, such as vocation imaging band specific cameras, visual band cameras, microphones, and the like.

In some embodiments, the vocational mask 130 may include an audio visual display, such as a stereo speaker, a virtual retinal display, a liquid crystal display, a virtual reality headset, and the like. A virtual retinal display may be a retinal scan display or retinal projector that draws a raster display directly onto the retina of the eye. In some embodiments, the virtual retinal display may include drive electronics that transmit data to a photon generator and/or intensity modulator. These components may process the data (e.g., video, audio, haptic, etc.) and transmit the processed data to a beam scanning component that further transmits data to an optical projector that projects an image and/or video to a retina of a user.

In some embodiments, the vocational mask 130 may include a network interface card that enables bidirectional communication (digital communication) with other vocational masks and/or computing device 140.

In some embodiments, the vocational mask 130 may provide a user interface to the user via the display described herein.

In some embodiments, the edge processor 132.2 may include a network interface card that enables digital communication with the vocational mask 130, the computing device 140, the cloud-based computing system 116, or the like.

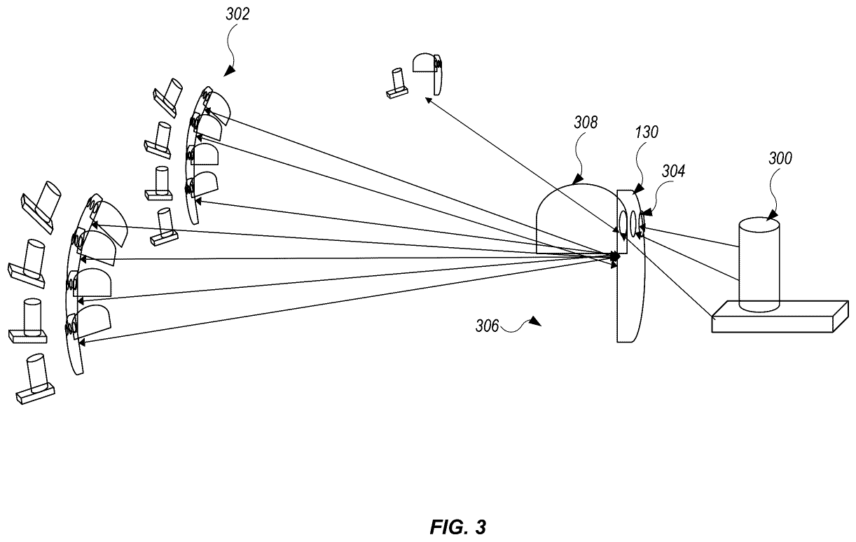

FIG. 3 illustrates bidirectional communication between communicatively coupled vocational masks 130 according to certain embodiments of this disclosure. As depicted, a user 306 is wearing a vocational mask 130 and 302. In the depicted example, the vocational mask 130 is attached to or integrated with a welding helmet 308. The user is viewing an object 300 (e.g., a workpiece). The vocational mask 130 may include multiple electromagnetic spectrum and/or acoustic sensors/imagers 304 to enable obtaining audio, video, acoustic, etc. data while observing the object 300 and/or performing a task (e.g., welding).

Further, as depicted, the vocational mask 130 may be communicatively coupled to one or more other vocational masks 302 worn by other users and may communicate data in real-time or near real-time such that bidirectional audio visual and haptic communications fosters a master-apprentice relationship, a teacher-student relationship, a relationship between collaborators, or other type of relationship. In some embodiments, the bidirectional communication enabled by the vocational masks 130 may enable collaboration between a teacher or collaborator and students. Each of the users wearing the vocational mask 130 may be enabled to visualize the object 300 that the user is viewing in real-time or near real-time.

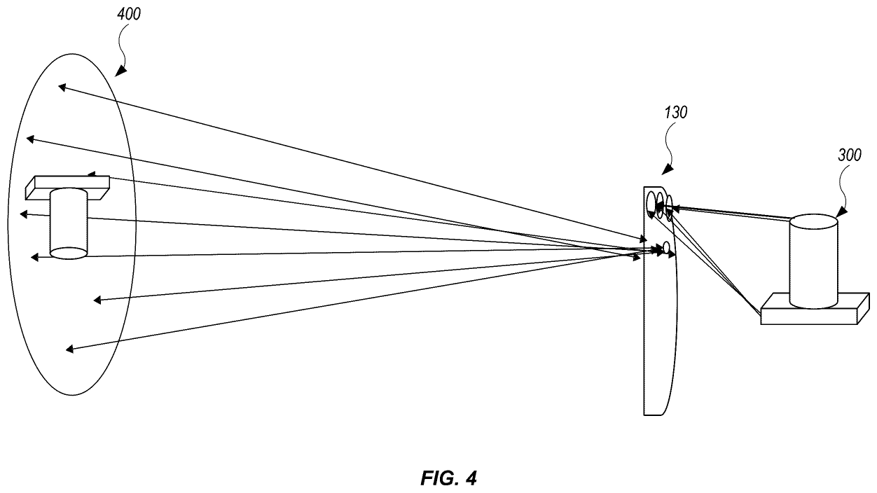

FIG. 4 illustrates an example of projecting an image onto a user's retina 400 via a virtual retinal display of a vocational mask 130 according to certain embodiments of this disclosure. As depicted, the imagers and/or cameras of the vocational mask 130 receive data pertaining to the object and the vocational mask 130 processes the data and projects an image representing the object 300 using a virtual retinal display onto the user's retina 400. The bidirectional communication with other users (e.g., students, master user, collaborator, teacher, supervisor, etc.) may enable projecting the image onto their retinas if they are wearing a vocational mask, as well. In some embodiments, the image may be displayed via a computing device 140 if the other users are not wearing vocational masks.

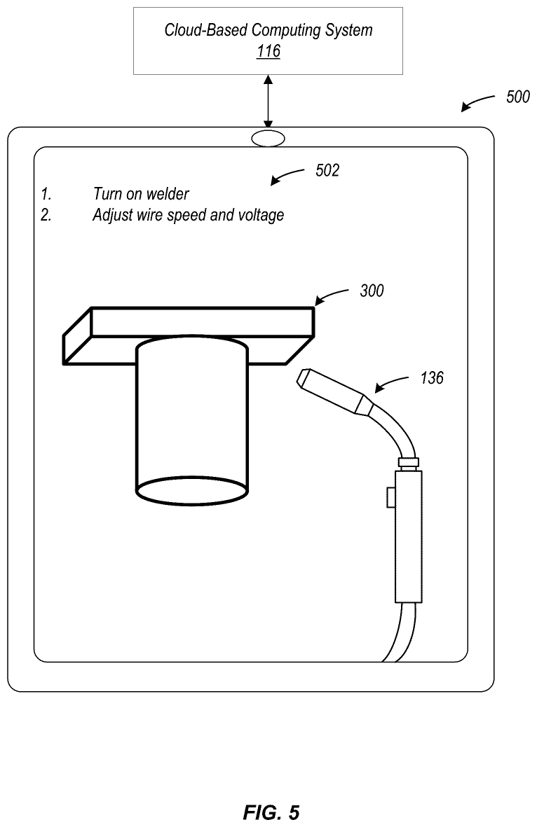

FIG. 5 illustrates an example of an image including instructions projected via a virtual retinal display of a vocational mask 130 according to certain embodiments of this disclosure. The example user interface 500 depicts actual things the user is looking at, such as a tool 136 and an object 300 (e.g., a workpiece), through the vocational mask 130. Further, the user interface depicts instructions 502 pertaining to performing a task. The instructions 502 may be generated by one or more machine learning models 154 of the AI agent, or may be provided via a computing device 140 and/or other vocational mask being used by another user (e.g., master user, collaborator, teacher, supervisor, etc.). In the depicted example, the instructions 502 instruct the user to “1. Turn on welder; 2. Adjust wire speed and voltage”. The instructions 502 may be projected on the user's retina via the virtual retinal display and/or presented on a display of the vocational mask 130.

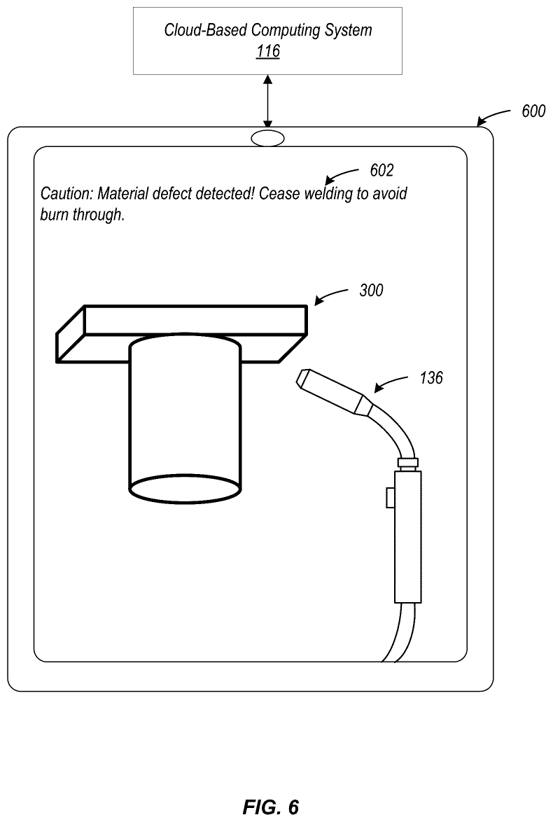

FIG. 6 illustrates an example of an image including a warning projected via a virtual retinal display of a vocational mask according to certain embodiments of this disclosure. The example user interface 600 depicts actual things the user is looking at, such as a tool 136 and an object 300, through the vocational mask 130. Further, the user interface depicts a warning 602 pertaining to performing a task. The warning 602 may be generated by one or more machine learning models 154 of the AI agent, or may be provided via a computing device 140 and/or other vocational mask being used by another user (e.g., master user, collaborator, teacher, supervisor, etc.). In the depicted example, the warning 602 indicates “Caution: Material defect detected! Cease welding to avoid burn through”. The warning 602 may be projected on the user's retina via the virtual retinal display and/or presented on a display of the vocational mask 130.

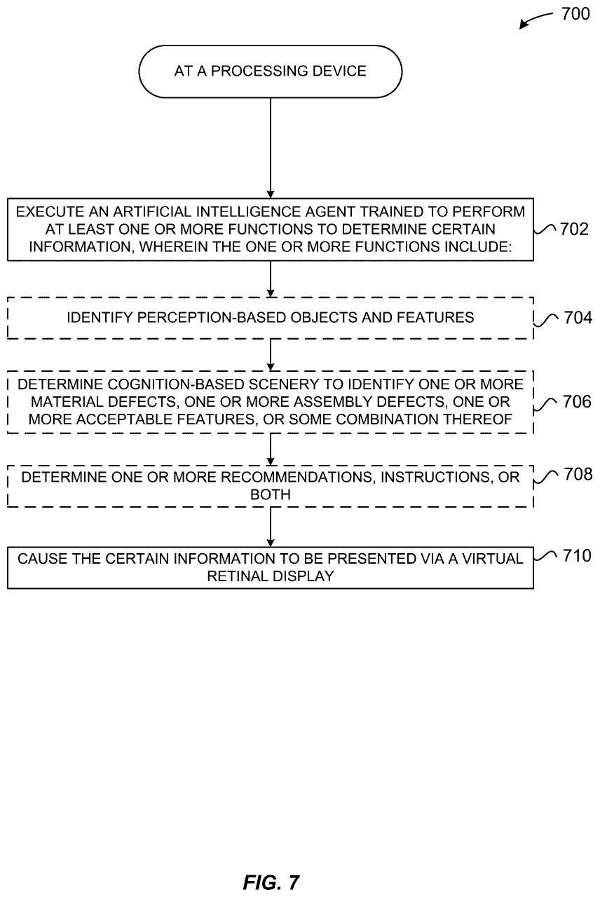

FIG. 7 illustrates an example of a method 700 for executing an artificial intelligence agent to determine certain information projected via a vocational mask of a user according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processing devices of a computing device (e.g., any component (server 128, training engine 152, machine learning models 154, etc.) of cloud-based computing system 116, vocational mask 130, edge processor 132 (132.1, 132.2), peripheral haptic device 134, tool 136, and/or computing device 140 of FIG. 1) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

For simplicity of explanation, the method 700 is depicted and described as a series of operations. However, operations in accordance with this disclosure can occur in various orders or concurrently, and with other operations not presented and described herein. For example, the operations depicted in the method 700 may occur in combination with any other operation of any other method disclosed herein. Furthermore, not all illustrated operations may be required to implement the method 700 in accordance with the disclosed subject matter. In addition, those skilled in the art will understand and appreciate that the method 700 could alternatively be represented as a series of interrelated states via a state diagram or events.

In some embodiments, one or more machine learning models may be generated and trained by the artificial intelligence engine and/or the training engine to perform one or more of the operations of the methods described herein. For example, to perform the one or more operations, the processing device may execute the one or more machine learning models. In some embodiments, the one or more machine learning models may be iteratively retrained to select different features capable of enabling optimization of output. The features that may be modified may include a number of nodes included in each layer of the machine learning models, an objective function executed at each node, a number of layers, various weights associated with outputs of each node, and the like.

In some embodiments, a system may include the vocational mask 130, which may include one or more virtual retinal displays, memory devices, processing devices, and other components as described herein. The processing devices may be communicatively coupled to the memory devices that store computer instructions, and the processing devices may execute the computer instructions to perform one or more of the steps of the method 700. In some embodiments, the system may include a welding helmet and the vocational mask may be coupled to the welding helmet. In some embodiments, the vocational mask may be configured to operate across both visible light and high intensity ultraviolet light conditions. In some embodiments, the vocational mask may provide protection against welding flash. In some embodiments, the vocational mask may be integrated with goggles. In some embodiments, the vocational mask may be integrated with binoculars or a monocular.

At block 702, the processing device may execute an artificial intelligence agent trained to perform at least one or more functions to determine certain information. The functions may include (i) identifying perception-based objects and features 704, (ii) determining cognition-based scenery to identify one or more material defects, one or more assembly defects, one or more acceptable features, or some combination thereof 706, and (iii) determining one or more recommendations, instructions, or both 708.

The artificial intelligence agent may include one or more machine learning models 154 trained to perform the functions. For example, at block 704, one or more machine learning models 154 may be trained to (i) identify perception-based objects and features using training data that includes labeled inputs of images including certain objects and features mapped to labeled outputs of identities or characterizations of those objects and features. The machine learning models may be trained to analyze aspects of the objects and features to compare the aspects to known aspects associated with known objects and features, and the machine learning models may perceive the identity of the analyzed objects and features. In other embodiments, one or more machine learning models 154 may be trained to (i) identify sound signatures (e.g., frequencies, amplitudes, etc.) associated with a burn through being reached or approaching using training data that includes labeled inputs of audio segments including certain burn through events mapped to labeled outputs of identities or characteristics of those outputs and features. The machine learning models may be trained to analyze aspects of the sound to compare the aspects to known aspects associated with known weld characteristics (e.g., burn through).

As indicated at block 706, the one or more machine learning models 154 may also be trained to (ii) determine cognition-based scenery or audio-based cues to identify one or more material defects, one or more assembly defects, one or more acceptable features, one or more work characteristics such as burn through, or some combination thereof using training data that includes labeled input of scenery images or audio of objects including material defects, assembly defects, and/or acceptable features mapped to labeled outputs that characterize and/or identify the material defects, assembly defects, work characteristics, and/or acceptable features. For example, one scenery image may include a portion of a submarine that includes parts that are welded together, and the machine learning models may be trained to cognitively analyze the scenery image to identify one or more portions of the scenery image that includes a welded part with a material welding defect, a part assembly defect, and/or acceptable welded feature. In embodiments, one audio segment may include sound that indicates a burn through, and the machine learning models may be trained to cognitively analyze the audio segments to identify one or more portions of audio segments that indicates a burn through.

As indicated at block 708, the one or more machine learning models 154 may be trained to (iii) determine one or more recommendations, instructions, or both using training data including labeled input of images (e.g., objects, products, tools, actions, etc.) and tasks to be performed (e.g., weld, grind, chamfer, debur, sand, polish, coat, etc.)

mapped to labeled outputs including recommendations, instructions, or both. The processing device may provide (e.g., via the virtual retinal display, a speaker, etc.) images, video, and/or audio that points out the defects and provides instructions, drawings, and/or information pertaining to how to fix the defects.

In addition, the output from performing one of the functions (i), (ii), and/or (iii) may be used as input to the other functions to enable the machine learning models 154 to generate a combined output. For example, the machine learning models 154 may identify a defect (a gouge) and provide welding instructions on how to fix the defect by filling the gouge properly via the vocational mask 130. Further, in some instances, the machine learning models 154 may identify several potential actions that the user can perform to complete the task and may aid the user's decision making by providing the actions in a ranked order of most preferred action to least preferred action or a ranked order of the action with the highest probability of success to the action with the lowest probability of success. In some embodiments, the machine learning models 154 may identify an acceptable feature (e.g., properly welded parts) and may output a recommendation to do nothing.

The artificial intelligence agent may further monitor one or more aspects of a virtual reality session. Based on the monitoring, the artificial intelligence agent may generate and provide, in real time, direction for performing a given task, during the virtual reality session, by a first user wearing the vocational mask. Alternatively, the artificial intelligence agent may generate suggested instructions by a master/teacher sharing the virtual reality session with an apprentice/student.

Embodiments are further contemplated in which the artificial intelligence agent may generate and provide, to various users of the shared virtual reality session, information in furtherance of completing a task where the various users are collaborators. In such collaborative environments, the various users of the shared virtual reality session may have different contexts, with such contexts being switchable among users. For example, a context may be control of a machine that can be carried out locally or remotely, and these contexts may be exchanged among the different users in the shared virtual reality session. The artificial intelligence agent may adjust which user receives particular information based on such context switches.

At block 710, the processing device may cause the certain information to be presented via the virtual retinal display. In some embodiments, the virtual retinal display may project an image onto at least one iris of the user to display alphanumeric data, graphic instructions, animated instructions, video instructions, or some combination thereof. In some embodiments, the vocational mask may include a stereo speaker to emit audio pertaining the information. In some embodiments, the processing device may superposition the certain information on a display (e.g., virtual retinal display).

In some embodiments, the vocational mask may include a network interface configured to enable bidirectional communication with a second network interface of a second vocational mask. The bidirectional communication may enable transmission of real-time or near real-time audio and video data, recorded audio and video data, or some combination thereof. “Real-time” may refer to less than 2 seconds and “near real-time” may refer to between 2 and 20 seconds.

In some embodiments, in addition to the vocational mask, a system may include a peripheral haptic device. The vocational mask may include a haptic interface, and the haptic interface may be configured to perform bidirectional haptic sensing and stimulation using the peripheral haptic device and the bidirectional communication. The stimulation may include precise mimicking, vibration, and the like. For example, the stimulation may include performing mimicked gestures via the peripheral haptic device. In other words, a master user may be using a peripheral haptic device to perform a task and the gestures performed by the master user using the peripheral haptic device may be mimicked by the peripheral haptic device being used by an apprentice user. In such a way, the master user may train and/or guide the apprentice user how to properly perform a task (e.g., weld) using the peripheral haptic devices.

The haptic interface may be communicatively coupled to the processing device. The haptic interface may be configured to sense, from the peripheral haptic device, hand motions, texture, temperature, vibration, slipperiness, friction, wetness, pulsation, stiction, friction, and the like. For example, the haptic interface may detect keystrokes when a user uses a virtual keyboard presented via the vocational mask using a display (e.g., virtual retinal display).

Further, the bidirectional communication provided by the vocational mask(s) and/or computing devices may enable a master user of a vocational mask and/or computing device to view and/or listen to the real-time or near real-time audio and video data, recorded audio and video data, or some combination thereof, and to provide instructions to the user via the vocational mask being worn by the user. In some embodiments, the bidirectional communication provided by the vocational mask(s) and/or computing devices may enable the user of a vocational mask and/or computing device to provide instructions to a set of students and/or apprentices via multiple vocational masks being worn by the students and/or apprentices. This technique may be beneficial for a teacher, collaborator, master user, and/or supervisor that is training the set of students.

In some embodiments, the user wearing a vocational mask may communicate with one or more users who are not wearing a vocational mask. For example, a teacher and/or collaborator may be using a computing device (e.g., smartphone) to see what a student is viewing and hear what the student is hearing using the bidirectional communication provided by the vocational mask worn by the student. The bidirectional communication provided by the vocational mask may enable a teacher or collaborator to receive, using a computing device, audio data, video data, haptic data, or some combination thereof, from the vocational mask being used by the user.

Additionally, the teacher and/or collaborator may receive haptic data, via the computing device, from the vocational mask worn by the student. The teacher and/or collaborator may transmit instructions (e.g., audio, video, haptic, etc.), via the computing device, to the vocational mask to guide and/or teach the student how to perform the task (e.g., weld) in real-time or near real-time.

In another example, the bidirectional communication may enable a user wearing a vocational mask to provide instructions to a set of students via a set of computing devices (e.g., smartphones). In this example, the user may be a teacher or collaborator and may be teaching a class or lesson on how to perform a task (e.g., weld) while wearing the vocational mask.

In some embodiments, the vocational mask may include one or more sensors to provide information related to geographical position, pose of the user, rotational rate of the user, or some combination thereof. In some embodiments, a position sensor may be used to determine a location of the vocational mask, an object, a peripheral haptic device, a tool, etc. in a physical space. The position sensor may determine an absolute position in relation to an established reference point. In some embodiments, the processing device may perform physical registration of the vocational mask, an object being worked on, a peripheral haptic device, a tool (e.g., welding gun, sander, grinder, etc.), etc. to map out the device in an environment (e.g., warehouse, room, underwater, etc.) in which the vocational mask, the object, the peripheral haptic device, etc. is located.

In some embodiments, the vocational mask may include one or more sensors including vocation imaging band specific cameras, visual band cameras, stereo microphones, acoustic sensors, or some combination thereof. The acoustic sensors may sense welding clues based on audio signatures associated with certain defects or issues, such as burn through. Machine learning models 154 may be trained using inputs of labeled audio signatures, labeled images, and/or labeled videos mapped to labeled outputs of defects. The artificial intelligence agent may process received sensor data, such as images, audio, video, haptics, etc., identify an issue (e.g., defect), and provide a recommendation (e.g., stop welding due to detected potential burn through) via the vocational mask.

In some embodiments, the vocational mask may include an optical bench that aligns the virtual retinal display to one or more eyes of the user.

In some embodiments, the processing device is configured to record the certain information, communications with other devices (e.g., vocational masks, computing devices), or both. The processing device may store certain information and/or communications as data in the memory device communicatively coupled to the processing device, and/or the processing device may transmit the certain information and/or communications as data feeds to the cloud-based computing system 116 for storage.

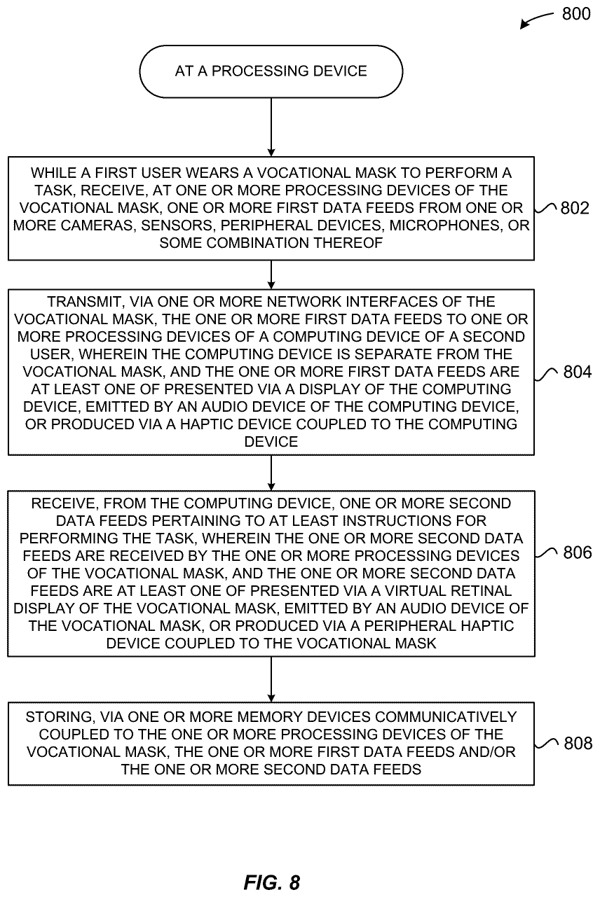

FIG. 8 illustrates an example of a method 800 for transmitting instructions for performing a task via bidirectional communication between a vocational mask and a computing device according to certain embodiments of this disclosure. The method 800 may be performed by processing logic that may include hardware (circuitry, dedicated logic, etc.), software, or a combination of both. The method 800 and/or each of their individual functions, subroutines, or operations may be performed by one or more processing devices of a computing device (e.g., any component (server 128, training engine 152, machine learning models 154, etc.) of cloud-based computing system 116, vocational mask 130, edge processor 132 (132.1, 132.2), peripheral haptic device 134, tool 136, and/or computing device 140 of FIG. 1) implementing the method 700. The method 800 may be implemented as computer instructions stored on a memory device and executable by the one or more processors. In certain implementations, the method 800 may be performed by a single processing thread. Alternatively, the method 800 may be performed by two or more processing threads, each thread implementing one or more individual functions, routines, subroutines, or operations of the methods.

For simplicity of explanation, the method 800 is depicted and described as a series of operations. However, operations in accordance with this disclosure can occur in various orders or concurrently, and with other operations not presented and described herein. For example, the operations depicted in the method 800 may occur in combination with any other operation of any other method disclosed herein. Furthermore, not all illustrated operations may be required to implement the method 800 in accordance with the disclosed subject matter. In addition, those skilled in the art will understand and appreciate that the method 800 could alternatively be represented as a series of interrelated states via a state diagram or events.

In some embodiments, one or more machine learning models may be generated and trained by the artificial intelligence engine and/or the training engine to perform one or more of the operations of the methods described herein. For example, to perform the one or more operations, the processing device may execute the one or more machine learning models. In some embodiments, the one or more machine learning models may be iteratively retrained to select different features capable of enabling optimization of output. The features that may be modified may include a number of nodes included in each layer of the machine learning models, an objective function executed at each node, a number of layers, various weights associated with outputs of each node, and the like.

At block 802, while a first user wears a vocational mask 130 to perform a task, the processing device may receive, at one or more processing devices of the vocational mask 130, one or more first data feeds from one or more cameras of the vocational mask 130, sensors of the vocational mask 130, peripheral haptic devices associated with the vocational mask 130, microphones of the vocational mask 130, or some combination thereof. In some embodiments, the vocational mask 130 may be attached to or integrated with a welding helmet and the task may be welding. In some embodiments, the task may be sanding, grinding, polishing, deburring, chamfering, coating, etc. The vocational mask 130 may be attached to or integrated with a helmet, a hat, goggles, binoculars, a monocular, or the like.

In some embodiments, the one or more first data feeds may include information related to video, images, audio, hand motions, haptics, texture, temperature, vibration, slipperiness, friction, wetness, pulsation, or some combination thereof. In some embodiments, the one or more first data feeds may include geographical position of the vocational mask 130, and the processing device may map, based on the geographical positon, the vocational mask 130 in an environment or a physical space in which the vocational mask 130 is located.

At block 804, the processing device may transmit, via one or more network interfaces of the vocational mask 130, the one or more first data feeds to one or more processing devices of the computing device 140 of a second user. In some embodiments, the computing device 140 of the second user may include one or more vocational masks, one or more smartphones, one or more tablets, one or more laptop computers, one or more desktop computers, one or more servers, or some combination thereof. The computing device 140 may be separate from the vocational mask 130, and the one or more first data feeds are at least one of presented via a display of the computing device 140, emitted by an audio device of the computing device 140, or produced or reproduced via a peripheral haptic device coupled to the computing device 140. In some embodiments, the first user may be an apprentice, student, trainee, or the like, and the second user may be a master user, a trainer, a teacher, a collaborator, a supervisor, or the like.

At block 806, the processing device may receive, from the computing device, one or more second data feeds pertaining to at least instructions for performing the task. The one or more second data feeds are received by the one or more processing devices of the vocational mask 130, and the one or more second data feeds are at least one of presented via a virtual retinal display of the vocational mask 130, emitted by an audio device (e.g., speaker) of the vocational mask 130, or produced or reproduced via a peripheral haptic device 134 coupled to the vocational mask 130.

In some embodiments, the instructions are presented, by the virtual retinal display of the vocational mask 130, via augmented reality. In some embodiments, the instructions are presented, by the virtual retinal display of the vocational mask, via virtual reality during a simulation associated with the task. In some embodiments, the processing device may cause the virtual retinal display to project an image onto at least one iris of the first user to display alphanumeric data associated with the instructions, graphics associated with the instructions, animations associated with the instructions, video associated with the instructions, or some combination thereof.

At block 808, the processing device may store, via one or more memory devices communicatively coupled to the one or more processing devices of the vocational mask 130, the one or more first data feeds and/or the one or more second data feeds.

In some embodiments, the processing device may cause the peripheral haptic device 134 to vibrate based on the instructions received from the computing device 140.

In some embodiments, the processing device may execute an artificial intelligence agent trained to perform at least one or more functions to determine certain information. The one or more functions may include (i) identifying perception-based objects and features, (ii) determining cognition-based scenery to identify one or more material defects, one or more assembly defects, one or more acceptable features, or some combination thereof, and (iii) determining one or more recommendations, instructions, or both.

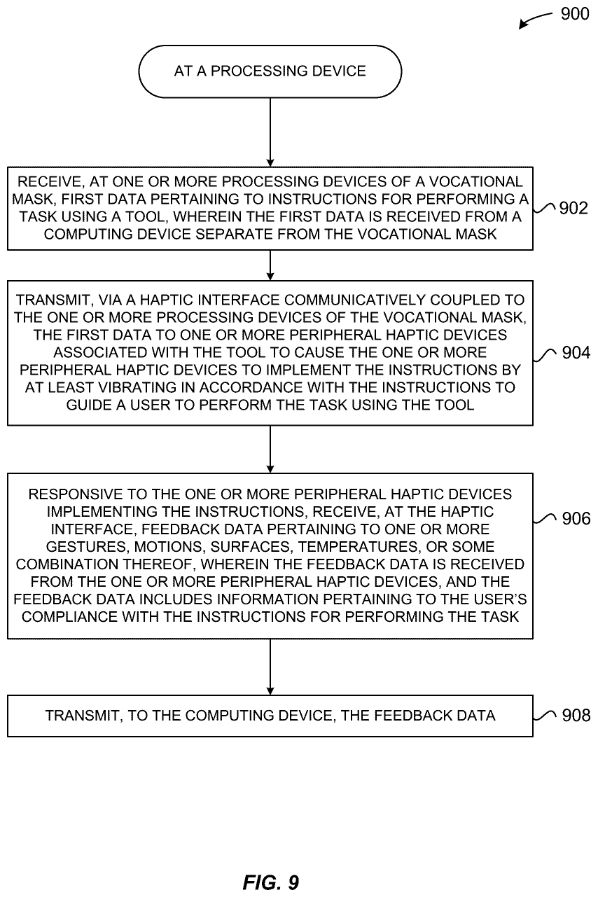

FIG. 9 illustrates an example of a method 900 for implementing instructions for performing a task using a peripheral haptic device according to certain embodiments of this disclosure. The method 900 may be performed by processing logic that may include hardware (circuitry, dedicated logic, etc.), software, or a combination of both. The method 900 and/or each of their individual functions, subroutines, or operations may be performed by one or more processing devices of a computing device (e.g., any component (server 128, training engine 152, machine learning models 154, etc.) of cloud-based computing system 116, vocational mask 130, edge processor 132 (132.1, 132.2), peripheral haptic device 134, tool 136, and/or computing device 140 of FIG. 1) implementing the method 900. The method 900 may be implemented as computer instructions stored on a memory device and executable by the one or more processors. In certain implementations, the method 900 may be performed by a single processing thread. Alternatively, the method 900 may be performed by two or more processing threads, each thread implementing one or more individual functions, routines, subroutines, or operations of the methods.

For simplicity of explanation, the method 900 is depicted and described as a series of operations. However, operations in accordance with this disclosure can occur in various orders or concurrently, and with other operations not presented and described herein. For example, the operations depicted in the method 900 may occur in combination with any other operation of any other method disclosed herein. Furthermore, not all illustrated operations may be required to implement the method 900 in accordance with the disclosed subject matter. In addition, those skilled in the art will understand and appreciate that the method 900 could alternatively be represented as a series of interrelated states via a state diagram or events.

In some embodiments, one or more machine learning models may be generated and trained by the artificial intelligence engine and/or the training engine to perform one or more of the operations of the methods described herein. For example, to perform the one or more operations, the processing device may execute the one or more machine learning models. In some embodiments, the one or more machine learning models may be iteratively retrained to select different features capable of enabling optimization of output. The features that may be modified may include a number of nodes included in each layer of the machine learning models, an objective function executed at each node, a number of layers, various weights associated with outputs of each node, and the like.

At block 902, the processing device may receive, at one or more processing devices of a vocational mask 130, first data pertaining to instructions for performing a task using a tool 136. The first data may be received from a computing device 140 separate from the vocational mask 130. In some embodiments, the computing device may include one or more peripheral haptic devices, one or more vocational masks, one or more smartphones, one or more tablets, one or more laptop computers, one or more desktop computers, one or more servers, or some combination thereof. In some embodiments, the task includes welding and the tool 136 is a welding gun.

At block 904, the processing device may transmit, via a haptic interface communicatively coupled to the one or more processing devices of the vocational mask 130, the first data to one or more peripheral haptic devices 134 associated with the tool 136 to cause the one or more peripheral haptic devices 134 to implement the instructions by at least vibrating in accordance with the instructions to guide a user to perform the task using the tool 136.

At block 906, responsive to the one or more peripheral haptic devices 134 implementing the instructions, the processing device may receive, from a haptic interface, feedback data pertaining to one or more gestures, motions, surfaces, temperatures, or some combination thereof. The feedback data may be received from the one or more peripheral haptic devices 134, and the feedback data may include information pertaining to the user's compliance with the instructions for performing the task.

At block 908, the processing device may transmit, to the computing device 140, the feedback data. In some embodiments, transmitting the feedback data may cause the computing device 140 to produce an indication of whether the user complied with the instructions for performing the task. The indication may be produced or generated via a display, a speaker, a different peripheral haptic device, or some combination thereof.

In some embodiments, in addition to the first data being received, video data may be received at the processing device of the vocational mask 130, and the video data may include video pertaining to the instructions for performing the task using the tool 136. In some embodiments, the processing device may display, via a virtual retinal display of the vocational mask 130, the video data. In some embodiments, the video data may be displayed concurrently with the instructions being implemented by the one or more peripheral haptic devices 134.

In some embodiments, in addition to the first data and/or video data being received, audio data may be received at the processing device of the vocational mask 130, and the audio data may include audio pertaining to the instructions for performing the task using the tool 136. In some embodiments, the processing device may emit, via a speaker of the vocational mask 130, the audio data. In some embodiments, the audio data may be emitted concurrently with the instructions being by the one or more peripheral haptic devices 134 and/or with the video data being displayed by the virtual retinal display. That is, one or more of video, audio, and/or haptic data pertaining to the instructions may be used concurrently to guide or instruct a user how to perform a task.

In some embodiments, in addition to the first data, video data, and/or audio data being received, virtual reality data may be received at the processing device of the vocational mask 130, and the virtual reality data may include virtual reality multimedia representing a simulation of a task. The processing device may execute, via at least a display of the vocational mask 130, playback of the virtual reality multimedia. For example, an artificial intelligent simulation generator may be configured to generate a virtual reality simulation for performing a task, such as welding an object using a welding gun. The virtual reality simulation may take into consideration various attributes, characteristics, parameters, and the like of the welding scenario, such as type of object being welded, type of welding, current amperage, length of arc, angle, manipulation, speed, and the like. The virtual reality simulation may be generated as multimedia that is presented via the vocational mask to a user to enable a user to practice, visualize, and experience performing certain welding tasks without actually welding anything.

In some embodiments, in addition to the first data, video data, audio data, and/or virtual reality data being received, augmented reality data may be received at the processing device of the vocational mask 130, and the augmented reality data may include augmented reality multimedia representing at least the instructions (e.g., via text, graphics, images, video, animation, audio). The processing device may execute, via at least a display of the vocational mask 130, playback of the augmented reality multimedia.

In some embodiments, the processing device may execute an artificial intelligence agent trained to perform at least one or more functions to determine certain information. The one or more functions may include (i) identifying perception-based objects and features, (ii) determining cognition-based scenery to identify one or more material defects, one or more assembly defects, one or more acceptable features, or some combination thereof, and/or (iii) determining one or more recommendations, instructions, or both. In some embodiments, the processing device may display, via a display (e.g., virtual retinal display or other display), the objects and features, the one or more material defects, the one or more assembly defects, the one or more acceptable features, the one or more recommendations, the instructions, or some combination thereof.

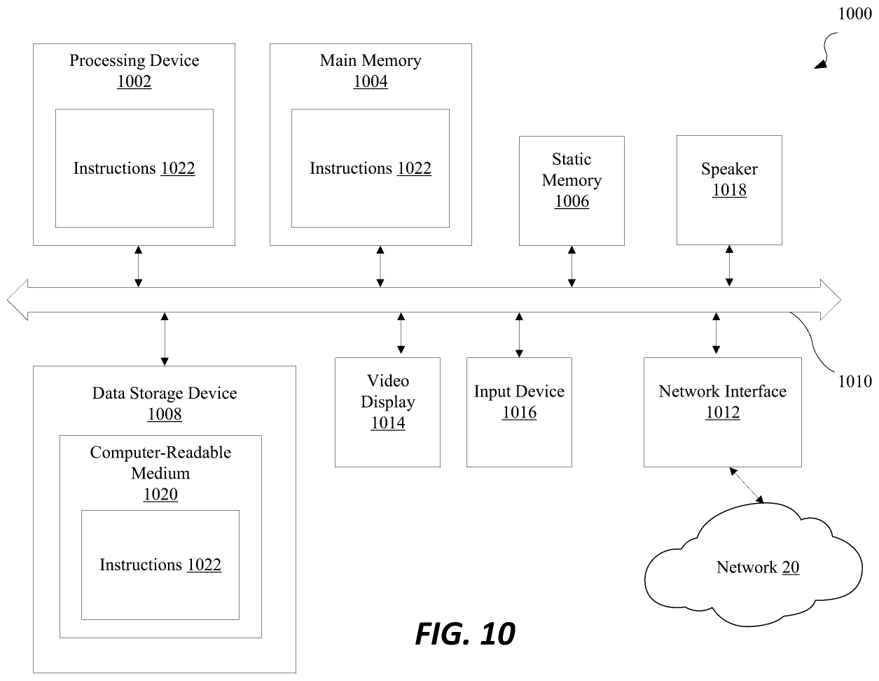

FIG. 10 illustrates an example computer system 1000, which can perform any one or more of the methods described herein. In one example, computer system 1000 may include one or more components that correspond to the vocational mask 130, the computing device 140, the peripheral haptic device 134, the tool 136, one or more servers 128 of the cloud-based computing system 116, or one or more training engines 152 of the cloud-based computing system 116 of FIG. 1. The computer system 1000 may be connected (e.g., networked) to other computer systems in a LAN, an intranet, an extranet, or the Internet. The computer system 1000 may operate in the capacity of a server in a client-server network environment. The computer system 1000 may be a personal computer (PC), a tablet computer, a laptop, a wearable (e.g., wristband), a set-top box (STB), a personal Digital Assistant (PDA), a smartphone, a smartwatch, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1000 includes a processing device 1002, a main memory 1004 (e.g., read-only memory (ROM), solid state drive (SSD), flash memory, dynamic random access memory (DRAM) such as synchronous DRAM (SDRAM)), a static memory 1006 (e.g., solid state drive (SSD), flash memory, static random access memory (SRAM)), and a data storage device 1008, which communicate with each other via a bus 1010.

Processing device 1002 represents one or more general-purpose processing devices such as a microprocessor, central processing unit, or the like. More particularly, the processing device 1002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1002 may also be one or more special-purpose processing devices such as an application specific integrated circuit (ASIC), a field programmable gate array (FPGA), a digital signal processor (DSP), network processor, or the like. The processing device 1002 is configured to execute instructions for performing any of the operations and steps of any of the methods discussed herein.

The computer system 1000 may further include a network interface device 1012. The computer system 1000 also may include a video display 1014 (e.g., a liquid crystal display (LCD) or a cathode ray tube (CRT)), one or more input devices 1016 (e.g., a keyboard and/or a mouse), and one or more speakers 1018 (e.g., a speaker). In one illustrative example, the video display 1014 and the input device(s) 1016 may be combined into a single component or device (e.g., an LCD touch screen).

The data storage device 1008 may include a computer-readable medium 1020 on which the instructions 1022 embodying any one or more of the methodologies or functions described herein are stored. The instructions 1022 may also reside, completely or at least partially, within the main memory 1004 and/or within the processing device 1002 during execution thereof by the computer system 1000. As such, the main memory 1004 and the processing device 1002 also constitute computer-readable media. The instructions 1022 may further be transmitted or received over a network 20 via the network interface device 1012.

While the computer-readable storage medium 1020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

In some examples, systems and methods of the present disclosure are configured to implement a virtual coach or avatar to provide instructions, encouragement, etc. to the user. For example, a user interface may be configured to display and project the virtual coach via the virtual retinal display of a vocational mask as described herein.

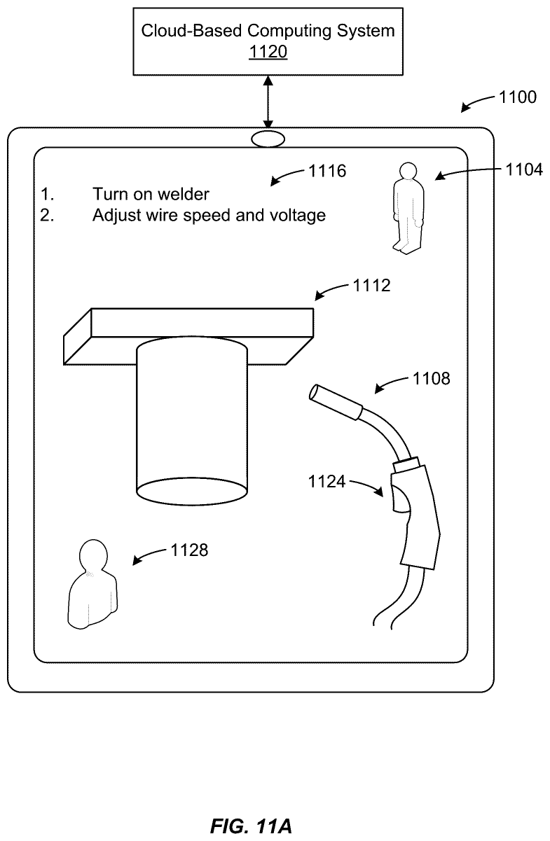

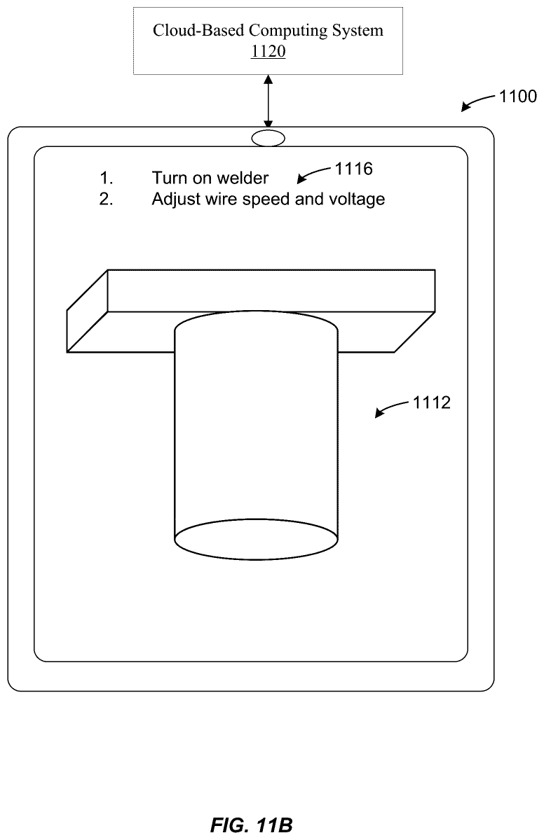

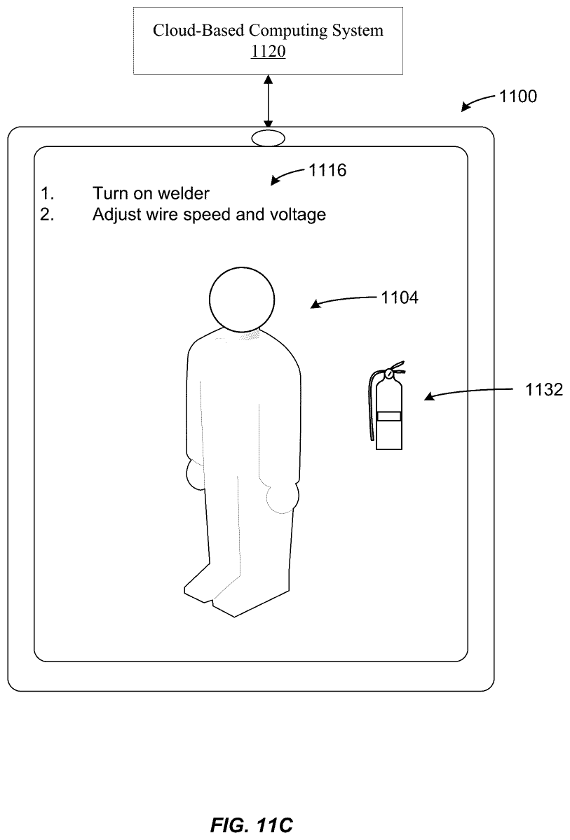

FIGS. 11A-11C show an example user interface 1100 configured to display a virtual avatar or coach 1104. The user interface 1100 presents an actual environment and objects (e.g., a tool 1108, a workpiece 1112, etc.) being viewed by the user through the vocational mask (e.g., through an eye opening, window, slot, etc. of the vocational mask 130) as well as projected graphical elements including, but not limited to, the virtual coach 1104, instructions 1116 pertaining to performing a task, etc. As described herein, the instructions 1116 may be generated by one or more machine learning models, or may be provided via one or more computing devices and/or other vocational masks being used by other users (e.g., master user, collaborator, teacher, supervisor, etc.). In some examples, the instructions 1116 and/or other commands or feedback, control signals, data, etc. may be provided to the user interface 1100 (e.g., to a computing device implementing the user interface 1100 at the vocational mask, such as the computing device 140) via a cloud-based computing system 1120.

The virtual coach 1104 is selected, generated, displayed, controlled, etc. based on one or more criteria as described below in more detail. For example, the virtual coach 1104 may have one or more personas (e.g., different professions, costumes or uniforms, characters, historical figures, etc., non-human personas such as animals, etc.), demeanors, appearances, voices, or other characteristics. Characteristics of the virtual coach 1104 may be modified based on user feedback and/or user performance (e.g., in real-time, based on historical performance over time, etc.). The virtual coach 1104 provides instructions via the user interface 1100 of the vocational mask as the user performs a work task (e.g., a welding task). For example, the instructions may be provided as text instructions (e.g., via a text bubble including the instructions 1116), voice instructions (e.g., audio instructions provided via speakers of the vocational mask as described herein), or combinations thereof.

Criteria for generating/controlling the virtual coach 1104 may include user characteristics, performance-based characteristics, task-based characteristics, or combinations thereof. For example, user characteristics may include, but are not limited to, user demographics or psychographics data, task-related physical characteristics (e.g., height, right-or-left-handedness, etc.), and so on. Performance-based characteristics may include, but are not limited to, historical performance data for the user and/or other users performing the same or similar task, real-time performance data collected as the user performs the current task, predictive data (e.g., generated by an ML model based on the historical performance data, the real-time performance data, etc.) indicative of predicted performance for a remainder of the current task, etc. Task-based characteristics correspond to characteristics of the specific task being performed and may include, but are not limited to, a type or category of the task (e.g., welding), an expected or average duration of the task (e.g., based on historical data), an amount of time the user has been performing the current task, an estimated amount of time remaining to perform the current task, a difficulty rating assigned to the task (e.g., on a numerical or other scale, such as a 1-10 scale), a number of times the user performed has performed the current task (e.g., in previous sessions), etc.

In some examples, the virtual coach 1104 is displayed in a fixed or static region of the user interface 1100. In other examples, a position of the virtual coach 1104 in the user interface 1100 is variable or dynamic. For example, the virtual coach 1104 may be displayed different regions of the user interface 1100 based on the task being performed, movement of the user, movement of the tool 1108, movement of the workpiece 1112, etc. As shown in FIG. 11A, the virtual coach 1104 is displayed in an unoccupied region of the user interface 1100 (e.g., a region of the user interface 1100 that does not include the tool 1108, the workpiece 1112, etc. As the user moves (e.g., causes the vocational mask to move, which in turn causes a field-of-view of the vocational mask to move), the virtual coach 1104 may move to a different, unoccupied region of the user interface 1100.

For example, the computing device may be configured to determine whether a current location of the virtual coach 1104 overlaps with other objects/graphical elements (e.g., occupies a same region as another object in an x-y coordinate system of the user interface 1100), identify an unoccupied region of the user interface 1100, and change the displayed location of the virtual coach 1104 to the unoccupied region. Similarly, a size of the virtual coach 1104 may be adjusted to fit within a particular unoccupied region of the user interface 1100. As shown in FIG. 11B, the workpiece 1112 substantially almost an entire field-of-view of the user interface 1100. Accordingly, in this example, the virtual coach 1104 may be omitted. Conversely, as shown in FIG. 11C, the workpiece 1112 may not be in the field-of-view of the user interface 1100. In this example, the virtual coach 1104 is enlarged and displayed in a central region of the user interface 1100.

In some examples, the virtual coach 1104 may be presented performing a same or similar task as the user (e.g., performing a welding task on a workpiece similar to the workpiece 1112, performing a welding task on a same portion of the workpiece 1112, welding along a same or similar path or welding line, etc.).

In various examples, movement/actions of the virtual coach 1104 may be automatically controlled by the computing device (e.g., AI- or ML model-controlled) and/or controlled/responsive to commands from a trainer, teacher, or other user as described herein. For example, when controlled by the computing device, the virtual coach 1104 may perform preset/predetermined movements or actions (e.g., a repeated movement, such as a looped animation, corresponding to a welding task being performed, which may demonstrate preferred motion or movement, speed, posture, etc. for the welding task). Conversely, when responsive to a trainer, the virtual coach 1104 may perform one of a plurality of possible movements or actions in response to commands from the trainer.

In still other examples, in some operating modes, the virtual coach 1104 may be controlled based on actual movements/actions of a trainer. For example, the trainer may selectively “inhabit” the virtual coach 1104 (e.g., from a remote location). In an example, the trainer may be presented with, on a display, a duplicate of the user interface 1104 to observe the actions of the user while performing the task. The trainer may be prompted to initiate or selectively initiate a demonstration mode in which the movement of the virtual coach 1104 is controlled by the movement of the trainer. For example, the trainer may perform a same or similar welding task on a workpiece or simply mimic preferred movements or postures for the welding task, which are then presented to the user in the user interface 1100 via movements of the virtual coach 1104. As an example, movement of the trainer may be monitored using various sensors as described herein, transmitted to the computing device associated with the vocational mask (e.g., via the cloud-based computing system 1120), and presented to the user via the virtual coach 1104.

In some examples, the appearance of the virtual coach 1104 may change based on whether the virtual coach 1104 is being controlled by the computing system or the trainer. For example, in response to the trainer taking control or inhabiting the virtual coach 1104, the appearance of the virtual coach 1104 may change to a persona associated with the trainer.

In some examples, one or more display-related characteristics of the virtual coach 1104 may vary based on performance of the task by the user. For example, to reduce a likelihood of distracting the user while performing a welding task, a determination of whether to display the virtual coach 1104, a size of the virtual coach 1104, movement of the virtual coach, etc. may be triggered based on one or inputs corresponding to actions performed by the user (e.g., based on whether the user is actively welding, as indicated by a welding button or trigger 1124 on the tool 1108 being actuated). For example, in response to the trigger 1124 being actuated, the virtual coach 1104 may be removed from the user interface 1104, a size of the virtual coach 1104 may be decreased, movement of the virtual coach 1104 may be stopped, volume of audio instructions may be decreased (or, in some examples, increased), etc.

Additionally or alternatively, a graphical element representing the user, such as a virtual avatar or user 1128, may be displayed via the user interface 1100. For example, the virtual user 1128 may mirror/imitate a posture, movements, etc. of the user. In some examples, the virtual coach 1104 may overlay the virtual user 1128 (e.g., the virtual coach 1104 may be at least partially transparent such that the virtual user 1128 is visible through the virtual coach 1104). In these examples, the virtual coach 1104 may be controlled to demonstrate a target or desired posture or movement. For example, the virtual coach 1104 may be represented as a looped or repeated animation demonstrating the task being performed by the user. Conversely a state or frame of animation of the virtual user 1128 may correspond to a current position or state of the actual user performing the welding task. According, the user can view a comparison between the user's movements/posture and the target or desired movements/posture.

Various other displayed characteristics of the virtual coach 1104 (and/or the virtual user 1128) may change during performance of the task by the user. For example, welding tasks are typically associated with challenges including, but are not limited to, user coordination (e.g., coordination of wire feeding, hand position, tool alignment, straightness of a weld, etc.), heat control (e.g., sufficient heat to perform the weld but maintaining heat below an amount that could damage the workpiece 1112), travel speed (e.g., travel speed of the tool 1108), and various safety considerations (heat, presence of fumes/gases, etc.). Accordingly, in some examples, characteristics of the virtual coach 1104 may change based on performance data indicative of one or more of user coordination, heat control, travel speed, and safety considerations. Other performance data may include, but is not limited to, data indicative of an amount of welding material used, thickness of a weld, porosity of a weld, penetration of a weld, and so on.

In one example, a displayed characteristic such as a color of clothing or other element, persona, expression or demeanor, size, position on the user interface 1100, etc. of the virtual coach 1104 may change based on the performance data. As one example, a color, persona, expression, etc. of the virtual coach 1104 may have two or more levels or tiers, such as a one level indicating satisfactory performance (e.g. measured indicator below a threshold) and another level indicating unsatisfactory performance (e.g., a measured indicator greater than or equal to the threshold). In an example, the displayed characteristic may include a first level indicating that the measured indicator is at least a first amount less than the threshold, a second level indicating that the measured indicator is approaching the threshold (e.g., within a certain range of the threshold), and a third level indicating that the measured indicator is greater than or equal to the threshold. The various levels may be represented by discrete states of the virtual coach 1104 and/or as a continuum or gradient.

For example, for user coordination, the measured indicator may correspond to a distance or amount of variation of an actual weld line from a target weld line and the threshold may correspond to a maximum allowable distance from the target weld line. For heat control, the measured indicator may correspond to a measured, sensed, estimated, or modeled heat value (e.g., based on inputs from sensors in the vocational mask, the tool 1108, in a vicinity of the workpiece 1112, etc.) and the threshold may include a first threshold corresponding to a minimum allowable heat and a second threshold corresponding to a maximum allowable heat. For travel speed, the measured indicator may correspond to a measured, sensed, estimated, or modeled speed (e.g., based on inputs from a sensor in the tool 1108 and/or attached to the user, a calculation based on observed movement of the tool 1108 within the user interface 1100, etc.) and the threshold may include a first threshold corresponding to a minimum allowable travel speed and a second threshold corresponding to a maximum allowable travel speed. For the various safety considerations, the measured indicator may correspond to measured, sensed, estimated, or modeled values of environmental indicators such as heat, gases, etc. and the threshold may correspond to a maximum measured amount or value.

In one example, a color of the virtual coach 1104 (e.g., a color of the entire graphical element corresponding to the virtual coach 1104, a color of clothing of the virtual coach 1104, etc.) may cycle through three possible colors (e.g., green, yellow, and red) in accordance with the first, second, and third levels as described above. In another example, an expression or demeanor of the virtual coach 1104 may cycle through three possible expressions, such as a happy or smiling expression, a neutral or concerned expression, and an angry or fearful expression. In another example, a posture of the virtual coach 1104 may cycle through three possible postures, such as a positive posture (e.g., a “thumbs up” gesture), a neutral or concerned posture (e.g., a wagging finger), and a negative posture (e.g., a “thumbs down” gesture).

In some examples, the changing or cycling may be the same for each display characteristic (e.g., the same possible colors for each level of heat control, travel speed, etc.) or may be different for each display characteristic. As one example, as shown in FIG. 11C, the cycling between levels may be accompanied by one or more icons 1132 (e.g., a fire extinguisher for heat control) or other graphical elements indicating which performance data is being indicated by the cycling display characteristic. As another example, each display characteristic may be associated with a particular type of performance data. For example, cycling expression may indicate different coordination levels, cycling color may indicate different heat control levels, cycling posture may indicate different safety consideration levels, etc. In this manner, various display characteristics of the virtual coach 1104 may dynamically change to provide different types of information to the user corresponding to different performance or other types of indicators. As used herein, in some contexts “display characteristics” may refer to audio characteristics, such as characteristics of a voice or vocal instructions associated with the virtual coach 1104, such as volume, tone, etc. of the voice.

Display characteristics of the virtual coach 1104 may change based on progress (e.g., a percentage or other measurement of a portion of the task that has been completed) and/or performance (e.g., one or more measures of performance, such as a calculated performance score or grade). For example, performance may be measured based on a comparison between performance of the current task and a baseline, reference, or target performance level of the task, a comparison between the performance of the current task and previous performances of the task by the same user or other users, etc. In an example, performance may be calculated (e.g., as a performance score) based on one or more performance values, which may correspond to measured or sensed values, calculated values, modeled or estimated values, or combinations thereof. As one example, a performance score may be based on a calculation of an overall amount of variation of performance values from one or more target values (e.g., a running average of performance values), an amount of times respective performance values varied (e.g., by at least a predetermined amount) from one or more target values, etc.

The performance values may be obtained using one or more techniques, including, but not limited to, imaging techniques (e.g., image processing of images displayed via the user interface 1100 to monitor and measure characteristics of the weld and work environment), acoustic sensing techniques (e.g., using one or more acoustic sensors as described herein), modeling techniques (e.g., techniques responsive to inputs from various sensors in the work environment, vocational mask, on or around the user, etc.), AI and ML techniques, and so on.

In an example, the performance score is calculated (e.g., using one or more of the ML models 154) based on a combination of performance values each corresponding to one or more of a plurality of measured, sensed, calculated, estimated, etc. values. For example, the performance score may be a numerical value, such as an integer or non-integer value (e.g., a value on a scale from 1 to 10, from 1 to 100, etc.) or a percentage. Each individual performance value may be calculated in accordance with a variation from a target value, where a maximum performance value (e.g., 100) is assigned for measurements within a minimum threshold of a target or baseline value and a minimum performance value (e.g., 0) is assigned for measurements greater than or equal to a maximum threshold of the target or baseline value. The target or baseline value may be a predetermined value (e.g., a predetermined target temperature), a dynamic value based on historical performance data (e.g., an average or other combination of temperatures measured during previous performances of the current task by the same or different users), or combinations thereof.

In an example, the performance score is an average or weighted average of the individual performance values. For example, one or more of the ML models 154 may be trained to obtain weights for each of the performance values based on assessments of overall weld quality (i.e., for a completed weld by the user and/or other users) and the various performance values calculated during performance of the respective welds. In other words, an ML model 154 may be trained to identify which performance values have the greatest effect on overall weld quality and assign weights accordingly. The weights may correspond to percentages or decimal values between 0 and 1, a ranking of the performance values (e.g., from 1 to n or from n to 1, where n is an integer greater than one), etc.

In this manner, the performance score may be continuously calculated and updated as the user performs the welding task and the virtual coach 1104 is generated and controlled based on the performance score (e.g., display characteristics such as colors, persona, demeanor, etc. of the virtual coach 1104 are changed based on the performance score). The display characteristics may be changed between discrete values or levels as described herein, changed in a gradient manner, etc.

In some examples, the virtual coach 1104 (e.g., display characteristics of the virtual coach 1104) may be controlled based in part on improvement or a rate of improvement (over time for the current task, a plurality of tasks, etc.) for the user and/or other users. For example, improvement may be measured based on improvement in the overall performance score and/or individual performance values for the current task, improvement from task-to-task, over time, for a plurality of tasks, etc. More specifically, various rates of improvement may be indexed to or correlated with (e.g., via an ML model) respective display characteristics and levels as described herein such that responsiveness to various display characteristics, for a given user or group of users, can be calculated. In other words, different users may show increased or decreased rates of improvement in response to different display characteristics of the virtual coach 1104 during specific stages or actions of a task. As one example, some users or types of users may be more responsive to higher levels (e.g., the third level described herein) and demonstrate greater rates of improvement while other users or types of users may be less responsive to higher levels and demonstrate lower rates of improvement or degraded performance. As described above, data indicative of improvement and/or rate of improvement may be referred to as “improvement data.”

Accordingly, the ML models according to the present disclosure may be trained to cycle through the various levels, omit or skip certain levels, use or not use certain colors or expressions, and change the display based on observed increases or decreases in performance, rates of improvement, etc. of various users over time. For example, training data may include labeled inputs (e.g., increased or decreased performance scores, rates of improvement, etc. of users over time) mapped to labeled outputs (e.g., various levels, colors or expressions, etc. to modify a display). The training data may be used to train one or more of the ML models.

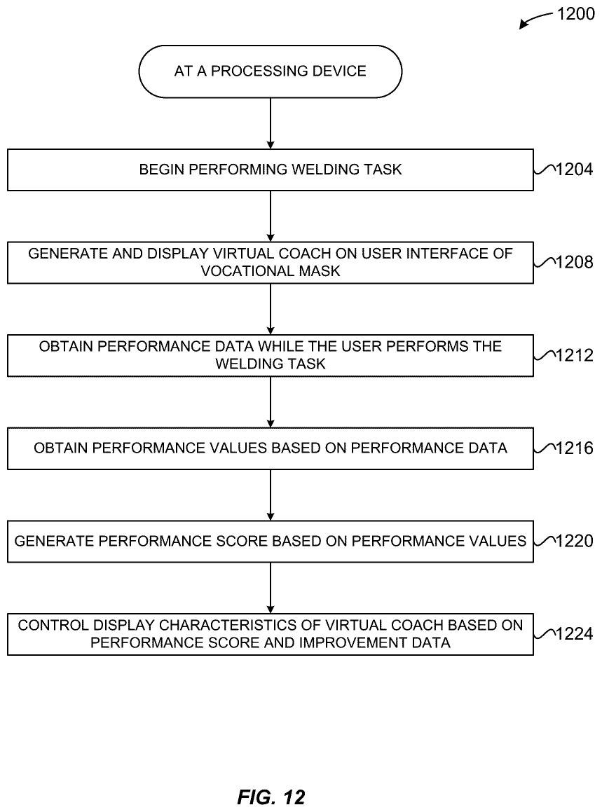

FIG. 12 illustrates steps of an example method 1200 of controlling a user interface and virtual coach according to embodiments of the present disclosure. One or more computing systems, computing devices, processors or processing devices, AI/ML models, etc. as described herein may be configured to perform various steps of the method 1200. While the method 1200 may be implemented for performing different types of tasks, the steps of the method 1200 are described with respect to a welding task for illustration purposes only.

At 1204, a user begins performing a welding task (e.g., on a workpiece.). Beginning to perform the welding task may include, but is not limited to, putting on a vocational mask as described herein and powering up and initializing welding equipment. In some examples, the user may log in or otherwise provide identification data (e.g., to a computing system) that includes or provides an indication of various user characteristics, previous performance data (e.g., historical data), user settings or preferences, etc. In some examples, beginning to perform the welding task may include providing an indication of the welding task, such as a type of welding task, a type of workpiece, etc. In some examples, the method 1200 includes, at 1204, obtaining data associated with the user and/or the welding task (e.g., from the database 129). The data may include, is not limited to, data identifying one or more characteristics of the user, historical data associated with performance of the welding task or other task by the user or other users, operating characteristics (e.g., target measurement or performance values) associated with the welding task or workpiece, etc.

At 1208, the method 1200 includes generating a virtual coach (and/or, in some examples, a virtual user) and displaying the virtual coach on a user interface of the vocational mask. Initially, the virtual coach may be a default (i.e., predetermined) virtual coach for the user or group of users, a virtual coach selected based on characteristics of the user, a virtual coach selected based on previous performances (e.g., welding sessions) of the user, etc.

At 1212, the method 1200 includes obtaining performance data while the user performs the welding task. The performance data may include measured, sensed, calculated, estimated, and/or modeled values as described herein. At 1216, the method 1200 includes obtaining performance values based on the performance data. The performance values may be calculated based on comparisons between the performance values and one or more respective reference, baseline, or target values as described herein.

At 1220, the method 1200 includes generating a performance score based on the performance data and/or performance values. For example, generating the performance score may include providing the performance values and/or performance data, historical performance data (e.g., for the user and/or other users), user characteristics (e.g., for the user and/or other users), or any other data described herein to one or more ML models trained to generate the performance score. In an example, the performance score is a weighted average of the performance values, and the one or more ML models are configured to calculate and assign weights to the performance values as described herein.

At 1224, the method 1200 includes controlling the virtual coach (e.g., controlling one or more display characteristics of the virtual coach) based on the performance score and, in some examples, further based on historical performance scores, values, and/or data for the user and/or other users. For example, the display characteristics may simply be controlled based on a comparison between the performance score and various ranges of performance scores (e.g., a determination of whether the performance score falls within a first range, a second range, a third range, etc.). As one example, a first range may be defined as a range of performance scores between 0 and m, a second range may be defined as a range between m and n, and a third range may be defined as a range between n and 100. The ranges may be same or different sizes, the ranges may be variable, and more or fewer ranges may be implemented.

In other examples, the display characteristics may be controlled based on comparisons between respective performance values and various ranges of performance values (e.g., comparisons between temperature values and ranges, travel speed values and ranges, etc.). In other words, in various examples, the display characteristics may be controlled based on an overall performance score (e.g., a performance score based on a combination of a plurality of performance values) and/or based on a plurality of different performance values corresponding to different measured (or calculated, modeled, etc.) values.

In other examples, controlling the virtual coach includes controlling the display characteristics further based on improvement data as described herein. For example, the method 1200 may include providing the performance score, performance data, etc. along with improvement data to an ML model trained to generate one or more virtual coach control outputs to control the display characteristics of the virtual coach. For example, rather than simply controlling the display characteristics based on the performance score and a comparison to various performance ranges as described above, the ML model may be configured to select/adjust various display characteristics further based on the improvement data. As one example, the performance ranges may be adjusted based on the improvement data (e.g., one or more performance ranges may be increased or decreased based on improvement data indicating that the user or other users or more likely or less likely to improve in response to specific display characteristics). As another example, one or more performance ranges may be eliminated. As another example, various display characteristics may be omitted (e.g., one or more colors, expressions, personas, etc. may not be displayed to a given user while other colors, expressions, personas, etc. are more likely to displayed to the user). As another example, audio characteristics may be adjusted (e.g., volume may be increased or decreased, voice or audio tones may be omitted or favored, etc.).

Accordingly, rather than simply controlling the display characteristics in the same manner for all users (or in the same manner for a given user for all tasks), the ML model is configured to generate control outputs based on improvement data indicative of which display characteristics are most effective for improving performance of a user or group of users.

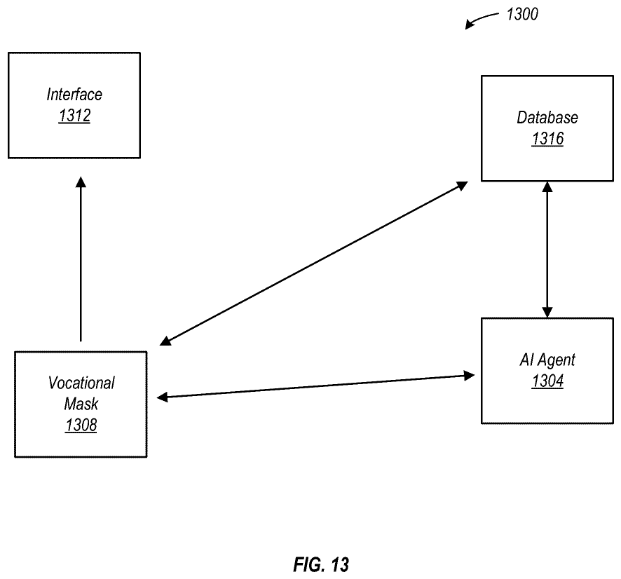

Various aspects of systems and methods described above, such as the virtual coach techniques described in FIGS. 11A, 11B, 11C, and 12, may be implemented using agentic AI techniques (e.g., an AI agent configured to implement agentic AI techniques to perform one or more functions associated with generation, control, etc. of a virtual coach). FIG. 13 is a block diagram illustrating one example of a system 1300 including an artificial intelligence (AI) agent 1304 configured to implement agentic AI techniques. The AI agent 1304 may be implemented by or one or more computing devices, such as one or more computing devices associated with a vocational mask 1308 having an interface 1312 (e.g., a user interface/display) as described herein. In some examples, one or more of the vocational masks 1308 and the AI agent 1304 may communicate with a database 1316. In some examples, the AI agent 1304 may be located/executed in a cloud computing system, such as cloud-based computing system 1120 described herein.

The AI agent 1304 receives various inputs, data, etc. from the vocational mask 1308 and the database 1316. For example, the AI agent 1304 may receive, from the vocational mask 1308, performance data associated with use of the vocational mask 1308 while a user performs a welding task, performance values, etc. as described above. The AI agent 1304 may further receive performance and/or other data associated use of other vocational masks (directly from the other vocational masks, from the database 1316, etc.). The AI agent 1304 is configured to implement various agentic AI techniques (e.g., autonomous or semi-autonomous, goal-oriented tasks or functions) to generate and control (i.e., control display characteristics of) the virtual coach based on the received data, generate instructions, etc. In some examples, the AI agent 1304 is configured to generate performance values and/or scores based on the performance data. In other examples, the AI agent 1304 is configured to receive the performance values and/or scores previously calculated by another entity (e.g., one or more of the vocational masks 1308, directly or via the database 1316).

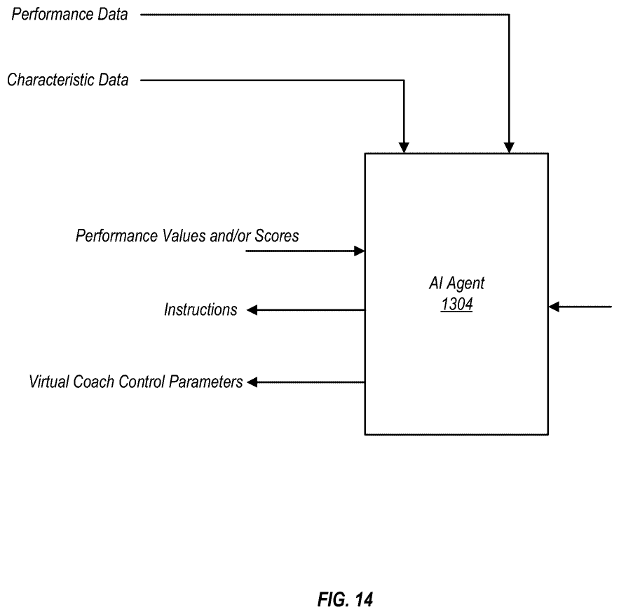

FIG. 14 is a block diagram of the example AI agent 1304. In the embodiment shown, the artificial intelligence agent 1304 may receive training information from a machine learning model. The training information may be used to determine how artificial intelligence agent 1304 evaluates the various inputs received during operation. For example, these inputs may include, but are not limited to: real-time, near real-time, or historical performance data; user, performance-based, display, and/or task-based characteristics (characteristic data); improvement data; trainer data (e.g., data associated with movement, actions, etc. of a trainer); etc.

Using the received inputs, the AI agent 1304 may generate and output virtual coach control parameters to control display characteristics of the virtual coach. For example the AI agent 1304 may generate control parameters as described above with respect to FIGS. 11A, 11B, 11C, and 12. In addition to the control parameters for the display characteristics, the AI agent 1304 may generate instructions for the user (e.g., via the interface, as described herein).

### Clauses

Clause 1. A system comprising:
a vocational mask configured to be worn by a user, wherein the vocational mask
comprises a user interface configured to present, to the user, one or more graphical elements associated with performing a work task; and
a computing device communicatively coupled to the user interface, wherein the computing device is configured to:
cause the user interface to display the one or more graphical elements, wherein the one or more graphical elements include a virtual coach,
receive performance data indicative of performance, by the user, of the work task while wearing the vocational mask, and
control at least one display characteristic of the virtual coach based on the performance data.

Clause 2. The system of any clause herein, wherein the user interface is configured to present the one or more graphical elements via a virtual retinal display.

Clause 3. The system of any clause herein, wherein the at least one display characteristic includes at least one of a persona, expression, posture, clothing, color, movement, size, and position of the virtual coach.

Clause 4. The system of any clause herein, wherein the computing device is configured to (i) obtain a performance score based on the performance data and (ii) control the at least one display characteristic based on the performance score.

Clause 5. The system of any clause herein, wherein the computing device is configured to (i) obtain a plurality of performance values based on the performance data and (ii) calculate the performance score based on the plurality of performance values.

Clause 6. The system of any clause herein, wherein the computing device is configured to (i) assign respective weights to the plurality of performance values and (ii) calculate the performance score as a weighted average of the performance values.

Clause 7. The system of any clause herein, wherein the computing device is configured to implement a machine learning model trained to calculate the performance score based on the performance data.

Clause 8. The system of any clause herein, wherein the machine learning model is configured to generate a control output to control the virtual coach based on the performance score and improvement data, and wherein the improvement data indicates improvement of performance of the user at the work task.

Clause 9. The system of any clause herein, wherein the work task is a welding task.

Clause 10. The system of any clause herein, wherein the performance data indicates at least one of a straightness of a weld, a thickness of the weld, temperature, travel speed, gas presence, an amount of welding material used, porosity of the weld, and penetration of the weld.

Clause 11. A computer-implemented method, comprising:
executing, by one or more computing devices communicatively coupled to a vocational mask configured to be worn by a user, instructions stored in memory, wherein the instructions include:
presenting, to the user via a user interface of the vocational mask, one or more graphical elements associated with performing a work task, wherein the one or more graphical elements include a virtual coach,
receiving performance data indicative of performance, by the user, of the work task while wearing the vocational mask, and
controlling at least one display characteristic of the virtual coach based on the performance data.

Clause 12. The method of any clause herein, wherein the user interface is configured to present the one or more graphical elements via a virtual retinal display.

Clause 13. The method of any clause herein, wherein the at least one display characteristic includes at least one of a persona, expression, posture, clothing, color, movement, size, and position of the virtual coach.

Clause 14. The method of any clause herein, wherein the instructions include (i) obtaining a performance score based on the performance data and (ii) controlling the at least one display characteristic based on the performance score.

Clause 15. The method of any clause herein, wherein the instructions include (i) obtaining a plurality of performance values based on the performance data and (ii) calculating the performance score based on the plurality of performance values.

Clause 16. The method of any clause herein, wherein the instructions include (i) assigning respective weights to the plurality of performance values and (ii) calculating the performance score as a weighted average of the performance values.

Clause 17. The method of any clause herein, wherein the instructions include executing a machine learning model trained to calculate the performance score based on the performance data.

Clause 18. The method of any clause herein, wherein the machine learning model is configured to generate a control output to control the virtual coach based on the performance score and improvement data, and wherein the improvement data indicates improvement of performance of the user at the work task.

Clause 19. The method of any clause herein, wherein the work task is a welding task.

Clause 20. The method of any clause herein, wherein the performance data indicates at least one of a straightness of a weld, a thickness of the weld, temperature, travel speed, gas presence, an amount of welding material used, porosity of the weld, and penetration of the weld.

Clause 21. A tangible, non-transitory computer-readable medium storing instructions that, when executed by a processing device, cause the processing device to perform functions comprising:
presenting, to a user via a user interface of a vocational mask worn by the user, one or more graphical elements associated with performing a work task, wherein the one or more graphical elements include a virtual coach;
receiving performance data indicative of performance, by the user, of the work task while wearing the vocational mask; and
controlling at least one display characteristic of the virtual coach based on the performance data.

Clause 22. The computer-readable medium of any clause herein, wherein the instructions include causing the user interface to present the one or more graphical elements via a virtual retinal display.

Clause 23. The computer-readable medium of any clause herein, wherein the at least one display characteristic includes at least one of a persona, expression, posture, clothing, color, movement, size, and position of the virtual coach.

Clause 24. The computer-readable medium of any clause herein, wherein the functions include (i) obtaining a performance score based on the performance data and (ii) controlling the at least one display characteristic based on the performance score.

Clause 25. The computer-readable medium of any clause herein, wherein the functions include (i) obtaining a plurality of performance values based on the performance data and (ii) calculating the performance score based on the plurality of performance values.

Clause 26. The computer-readable medium of any clause herein, wherein the functions include (i) assigning respective weights to the plurality of performance values and (ii) calculating the performance score as a weighted average of the performance values.

Clause 27. The computer-readable medium of any clause herein, wherein the functions include executing a machine learning model trained to calculate the performance score based on the performance data.

Clause 28. The computer-readable medium of any clause herein, wherein the functions include, using the machine learning model, generating a control output to control the virtual coach based on the performance score and improvement data, and wherein the improvement data indicates improvement of performance of the user at the work task.

Clause 29. The computer-readable medium of any clause herein, wherein the work task is a welding task, and wherein the performance data indicates at least one of a straightness of a weld, a thickness of the weld, temperature, travel speed, gas presence, an amount of welding material used, porosity of the weld, and penetration of the weld.

Clause 30. A vocational mask configured to be worn by a user, the vocational mask comprising:
a user interface configured to present, to the user via a virtual retinal display, one or more graphical elements associated with performing a welding task; and
a computing device communicatively coupled to the user interface, wherein the computing device is configured to:
cause the user interface to display the one or more graphical elements, wherein the one or more graphical elements include a virtual coach,
receive (i) performance data indicative of performance, by the user, of the welding task while wearing the vocational mask and (ii) improvement data correlating improvement of performance of the user at the welding task to a plurality of display characteristics of the virtual coach, and
control at least one display characteristic of the plurality of display characteristics based on the performance data.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

1. A system comprising:
a vocational mask configured to be worn by a user, wherein the vocational mask comprises a user interface configured to present, to the user, one or more graphical elements associated with performing a work task; and
a computing device communicatively coupled to the user interface, wherein the computing device is configured to:
cause the user interface to display the one or more graphical elements, wherein the one or more graphical elements include a virtual coach,
receive performance data indicative of performance, by the user, of the work task while wearing the vocational mask, and
control at least one display characteristic of the virtual coach based on the performance data.

2. The system of claim 1, wherein the user interface is configured to present the one or more graphical elements via a virtual retinal display.

3. The system of claim 1, wherein the at least one display characteristic includes at least one of a persona, expression, posture, clothing, color, movement, size, and position of the virtual coach.

4. The system of claim 1, wherein the computing device is configured to (i) obtain a performance score based on the performance data and (ii) control the at least one display characteristic based on the performance score.

5. The system of claim 4, wherein the computing device is configured to (i) obtain a plurality of performance values based on the performance data and (ii) calculate the performance score based on the plurality of performance values.

6. The system of claim 5, wherein the computing device is configured to (i) assign respective weights to the plurality of performance values and (ii) calculate the performance score as a weighted average of the performance values.

7. The system of claim 4, wherein the computing device is configured to implement a machine learning model trained to calculate the performance score based on the performance data.

8. The system of claim 7, wherein the machine learning model is configured to generate a control output to control the virtual coach based on the performance score and improvement data, and wherein the improvement data indicates improvement of performance of the user at the work task.

9. The system of claim 1, wherein the work task is a welding task.

10. The system of claim 9, wherein the performance data indicates at least one of a straightness of a weld, a thickness of the weld, temperature, travel speed, gas presence, an amount of welding material used, porosity of the weld, and penetration of the weld.

11. A computer-implemented method, comprising:
executing, by one or more computing devices communicatively coupled to a vocational mask configured to be worn by a user, instructions stored in memory, wherein the instructions include:
presenting, to the user via a user interface of the vocational mask, one or more graphical elements associated with performing a work task, wherein the one or more graphical elements include a virtual coach,
receiving performance data indicative of performance, by the user, of the work task while wearing the vocational mask, and
controlling at least one display characteristic of the virtual coach based on the performance data.

12. The method of claim 11, wherein the user interface is configured to present the one or more graphical elements via a virtual retinal display.

13. The method of claim 11, wherein the at least one display characteristic includes at least one of a persona, expression, posture, clothing, color, movement, size, and position of the virtual coach.

14. The method of claim 11, wherein the instructions include (i) obtaining a performance score based on the performance data and (ii) controlling the at least one display characteristic based on the performance score.

15. The method of claim 14, wherein the instructions include (i) obtaining a plurality of performance values based on the performance data and (ii) calculating the performance score based on the plurality of performance values.

16. The method of claim 15, wherein the instructions include (i) assigning respective weights to the plurality of performance values and (ii) calculating the performance score as a weighted average of the performance values.

17. The method of claim 14, wherein the instructions include executing a machine learning model trained to calculate the performance score based on the performance data.

18. The method of claim 17, wherein the machine learning model is configured to generate a control output to control the virtual coach based on the performance score and improvement data, and wherein the improvement data indicates improvement of performance of the user at the work task.

19. The method of claim 11, wherein the work task is a welding task.

20. The method of claim 19, wherein the performance data indicates at least one of a straightness of a weld, a thickness of the weld, temperature, travel speed, gas presence, an amount of welding material used, porosity of the weld, and penetration of the weld.

21. A tangible, non-transitory computer-readable medium storing instructions that, when executed by a processing device, cause the processing device to perform functions comprising:
presenting, to a user via a user interface of a vocational mask worn by the user, one or more graphical elements associated with performing a work task, wherein the one or more graphical elements include a virtual coach;
receiving performance data indicative of performance, by the user, of the work task while wearing the vocational mask; and
controlling at least one display characteristic of the virtual coach based on the performance data.

22. The computer-readable medium of claim 21, wherein the instructions include causing the user interface to present the one or more graphical elements via a virtual retinal display.

23. The computer-readable medium of claim 21, wherein the at least one display characteristic includes at least one of a persona, expression, posture, clothing, color, movement, size, and position of the virtual coach.

24. The computer-readable medium of claim 21, wherein the functions include (i) obtaining a performance score based on the performance data and (ii) controlling the at least one display characteristic based on the performance score.

25. The computer-readable medium of claim 24, wherein the functions include (i) obtaining a plurality of performance values based on the performance data and (ii) calculating the performance score based on the plurality of performance values.

26. The computer-readable medium of claim 25, wherein the functions include (i) assigning respective weights to the plurality of performance values and (ii) calculating the performance score as a weighted average of the performance values.

27. The computer-readable medium of claim 24, wherein the functions include executing a machine learning model trained to calculate the performance score based on the performance data.

28. The computer-readable medium of claim 27, wherein the functions include, using the machine learning model, generating a control output to control the virtual coach based on the performance score and improvement data, and wherein the improvement data indicates improvement of performance of the user at the work task.

29. The computer-readable medium of claim 21, wherein the work task is a welding task, and wherein the performance data indicates at least one of a straightness of a weld, a thickness of the weld, temperature, travel speed, gas presence, an amount of welding material used, porosity of the weld, and penetration of the weld.

30. A vocational mask configured to be worn by a user, the vocational mask comprising:
a user interface configured to present, to the user via a virtual retinal display, one or more graphical elements associated with performing a welding task; and
a computing device communicatively coupled to the user interface, wherein the computing device is configured to:
cause the user interface to display the one or more graphical elements, wherein the one or more graphical elements include a virtual coach,
receive (i) performance data indicative of performance, by the user, of the welding task while wearing the vocational mask and (ii) improvement data correlating improvement of performance of the user at the welding task to a plurality of display characteristics of the virtual coach, and
control at least one display characteristic of the plurality of display characteristics based on the performance data.
